(19) United States
(12) Patent Application Publication
(10) Pub. No.: US 2022/0234999 A1
(43) Pub. Date: Jul. 28, 2022

(54) METHOD FOR CATALYTICALLY PRODUCING UREA

(71) Applicants: thyssenkrupp Industrial Solutions AG, Essen (DE); thyssenkrupp AG, Essen (DE)

(72) Inventors: Yevgeny MAKHYNYA, Mülheim an der Ruhr (DE); Tarek EL HAWARY, Holzwickede (DE); Christoph GLOTZBACH, Speyer (DE); Nils TENHUMBERG, Dortmund (DE); Walter LEITNER, Aachen (DE); Jürgen KLANKERMAYER, Essen (DE); Hannah SCHUMACHER, köln (DE)

(73) Assignees: thyssenkrupp Industrial Solutions AG, Essen (DE); thyssenkrupp AG, Essen (DE)

(21) Appl. No.: 17/607,716

(22) PCT Filed: Apr. 27, 2020
(86) PCT No.: PCT/EP2020/061614

§ 371 Date: Oct. 29, 2021

(30) Foreign Application Priority Data
Apr. 29, 2019 (DE) 10 2019 111 058.0

(51) Int. Cl. C07C 273/02; C07C 231/10; C07C 273/10

(52) U.S. Cl. CPC C07C 273/02; C07C 273/10; C07C 231/10

(57) ABSTRACT

A process for preparing urea comprises preparing formamide based on carbon dioxide, hydrogen, and ammonia, forming methyl formate or ammonium formate as an intermediate in a catalytic reaction, and preparing urea by reacting the formamide and possibly ammonia in the presence of a catalyst. The source of carbon dioxide is a liquid laden with chemically and/or physically bound carbon dioxide and selected from a methanol phase or an aqueous ammonia solution obtained by gas scrubbing of a syngas for removing carbon dioxide using a scrubbing fluid. The scrubbing fluid can be a methanol phase, or carbon dioxide is desorbed from the scrubbing fluid and absorbed into a methanol phase to give a carbon dioxide-laden methanol phase that is then reacted as carbon dioxide-containing stream with a hydrogen-containing stream in the presence of a catalyst to form methyl formate. The methyl formate is reacted with an ammonia-containing stream to form formamide.

The invention relates to a process for the catalytic preparation of urea.

Urea, the diamide of carbonic acid, is one of the most important bulk chemicals and is used predominantly as fertilizer. As such it possesses a high nitrogen content (46 wt %). It is easily hydrolyzed, releasing ammonia and CO₂, by the enzyme urease, which is produced by microorganisms and occurs widely in the soil.

Furthermore, urea is an important building block for organic products, such as melamine, and a raw material for synthetic resins and fibers. It is used as a cattle feed additive and in the production of drugs and explosives, and in the textile industry as well. In recent decades, urea has also gained importance as a reducing agent for the NOx reduction of diesel exhaust gases.

The industrial production of urea is presently based almost exclusively on the high-pressure synthesis from ammonia (NH₃) and carbon dioxide (CO₂) at around 150 bar and around 180° C. The two reactants generally come from an ammonia plant, which is usually situated in the close vicinity of a urea plant. Ammonia is obtained industrially from nitrogen and hydrogen, the reactants being used in the form of a syngas, from which disruptive substances such as sulfur compounds or carbon dioxide are removed.

One significant approach to urea synthesis is based on what is called the carbamate route. In this case the carbon dioxide required as starting material is obtained by separation from a syngas produced for the ammonia synthesis. In the course of the operation, the CO₂ separated off in advance is brought into association with liquid ammonia. In the first step of the synthesis, ammonium carbamate is synthesized primarily. During the course of the reaction, urea as well is formed in small quantities, to produce a complex mixture composed of ammonia, CO₂, urea, ammonium carbamate, ammonium hydrogencarbonate, and water. This takes place in an apparatus referred to as a carbamate condenser. The reaction mixture departs the carbamate condenser for the urea reactor, where the actual urea formation reaction occurs.

Because the carbamate is a highly corrosive medium, especially at high temperatures and pressures, a specific, corrosion-resistant steel is required at many points in the process. One specialty steel suitable for this purpose is available commercially under the designation Safurex®. The Safurex steel is utilized as a material for the inner coatings of the apparatuses and for the piping, among other components. The acquisition/utilization of this steel is extremely costly and massively increases the capital costs of the plant. Not only the steel but also the high-pressure and high-temperature operation impose a major challenge for the apparatuses in the high-pressure circuit, this being ultimately reflected in the acquisition costs of these apparatuses. These disadvantages can be overcome by the present invention.

Substituted urea derivatives can be prepared catalytically via various routes, using CO and CO₂ or other carbonylating agents. The synthesis of substituted urea derivatives by means of CO₂ is described for example in P. Munshi, et al., Tetrahedron Lett. 2003, 44, 2725-2727. The synthesis with other carbonylating agents is reported for example in A. Basha, Tetrahedron Lett. 1988, 29, 2525-2526.

Relative to the incorporation of amines for substituted ureas, additional challenges arise when using ammonia to prepare urea, since ammonia has three potentially active hydrogens and a significantly different basicity. Consequently there are only relatively few publications which report on catalytic synthesis of urea, examples including F. Barzagli et al., Green Chem. 2011, 13, 1267-1274, M. M. Taqui Khan et al., J. Mol. Catal. 1988, 48, 25-27, and D. C. Butler et al., lnorg. Chem. Commun. 1999, 2, 305-307.

The inventors undertook the consideration of providing a process for the catalytic synthesis of urea on the basis of formamide as starting material, in order to overcome the disadvantages described above for the conventional processes.

The industrial production of formamide, also called methanamide or formic acid amide, is presently based almost exclusively on two synthetic routes, both based on carbon monoxide as C1 starting material and both using sodium methoxide as catalyst. The first route is that of the direct carbonylation of ammonia (NH₃) with carbon monoxide (CO) (eq. 1).

CO+NH₃→HCONH₂ (eq. 1)

The second route entails the reaction of carbon monoxide with methanol using sodium methoxide as catalyst to give methyl formate, with subsequent ammonolysis (eq. 2-3). Methanol liberated in this reaction is recycled.

CO+CH₃OH→HCOOCH₃ (eq. 2)

HCOOCH₃+NH₃→HCONH₂+CH₃OH (eq. 3)

Both routes employ CO. CO on the one hand is a very valuable reactant but on the other hand is also a highly toxic reactant, and is also decidedly complicated to produce.

An alternative to the utilization of CO is to utilize carbon dioxide (CO₂). The utilization of CO₂ for the C1 chemistry is highly attractive, particularly from the standpoints of safety and economics. CO₂ is one of the products from the steam reforming operation that is separated off from the process gas in a pure or near-pure state. It can be isolated in a state of chemical purity both from the flue gas and from the reformer gas.

The problem associated with the physical utilization of CO₂, however, lies in the thermodynamic stability of CO₂. The molecule is inert in the majority of chemical reactions. In unusual cases, however, it is possible to activate CO₂ by means of specific catalysts.

WO 2013/014160 A1 describes the formation of formamide from ammonia, carbon dioxide and hydrogen in methanol using a catalyst composed of 1% gold on TiO₂, and the formation of methyl formate from carbon dioxide and hydrogen in methanol using a catalyst composed of 1% gold on TiO₂ or Al₂O₃.

US 2012/071690 A1 describes the production of formamides via an ammonium formate intermediate. The ammonium formate intermediate is formed from carbon dioxide and hydrogen and a tertiary amine with the aid of a catalyst, preferably containing ruthenium, rhodium, palladium, osmium and/or iridium, which constitutes an adduct of formic acid and the tertiary amine. The adduct is subsequently isolated and converted with ammonia or amines into the formamide.

DE 102012019441 A1 describes the production of formamide with the aid of iridium catalysts from the reactants methanol, ammonia, carbon dioxide and hydrogen.

In the DTIC document AD-A199-861 from 1988, Vaska et al. describe the production of formamide from ammonia, carbon dioxide and hydrogen by means of the catalyst [Ir(Cl)(CO)(Ph₃P)₂].

These processes known from the prior art for producing formamide via the catalytic activation of CO₂ have the disadvantage that the yields of formamide are relatively low. Moreover, the catalysts used are quickly deactivated. Furthermore, a reaction time of several days may be necessary.

Ammonia is the usual starting material in the synthesis of urea. Furthermore, CO₂ is a readily available feedstock. In the search for a catalytic route to the synthesis of urea based on CO₂, the starting point contemplated was a two-stage process via formamide as intermediate, as depicted in scheme 1:

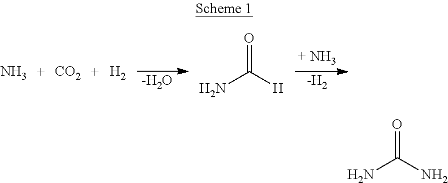

While the syntheses of substituted urea from formamides are described in the literature, as for example in S. Kotachi, Y. Tsuji, T. Kondo, Y. Watanabe, J. Chem. Soc., Chem. Commun. 1990, 549-550, the formation of urea from the reaction of formamide preferably with ammonia represents a new and challenging C—N bond formation. The problems with using CO₂, because of its thermodynamic stability, have been discussed above.

To summarize, existing solutions for the synthesis of urea are not satisfactory in all respects. Consequently there is a need for an alternative urea synthesis which avoids the disadvantages of the conventional urea syntheses. The feedstocks are to continue to originate, though not necessarily, from the ammonia synthesis.

The object on which the invention is based is that of providing an alternative process for producing urea, preferably based on feedstocks originating from the ammonia plant, which is suitable for industrial use and with which the requirements imposed on the plants—in relation, for example, to aggressiveness of substances formed and also to pressure and temperature conditions—can be reduced. It ought in particular to be possible to integrate the process conveniently into a customary ammonia synthesis plant, meaning that the feedstocks for the urea synthesis are to be able to be withdrawn from the process streams of an ammonia plant and to require at most slight modifications in the ammonia synthesis process. The main factor is that of extremely minimal intervention in the existing process design of the ammonia plant.

This object is achieved in accordance with the invention by means of a process as claimed in claim 1.

The object is achieved more particularly by a process for preparing urea that comprises:

a) preparing formamide on the basis of carbon dioxide, hydrogen and ammonia, with formation of methyl formate or ammonium formate as intermediate in a catalytic reaction, and
b) preparing urea by reacting the resultant formamide or the resultant formamide with ammonia in the presence of a catalyst,
where the source of carbon dioxide is a liquid laden with chemically and/or physically bound carbon dioxide and selected from a methanol phase or an aqueous ammonia solution which is obtained by gas scrubbing of a syngas for the removal of CO₂ using a scrubbing fluid, where
a1) the scrubbing fluid is a methanol phase, or CO₂ is desorbed from the scrubbing fluid laden with chemically and/or physically bound carbon dioxide and absorbed into a methanol phase to give a CO₂-laden methanol phase, and the CO₂-laden methanol phase is reacted as CO₂-containing stream with a hydrogen-containing stream in the presence of a catalyst to form methyl formate, and the resultant methyl formate is reacted with an ammonia-containing stream to form formamide, or
a2) the scrubbing fluid is an aqueous ammonia solution, and so CO₂ is bound at least partly in the form of carbonates in the scrubbing fluid, and this scrubbing fluid laden with chemically and/or physically bound CO₂ is reacted as CO₂-containing stream with a hydrogen-containing stream in the presence of a catalyst and optionally organic solvent to form ammonium formate or to form ammonium formate and formamide, and the resultant ammonium formate is converted into formamide by heat treatment.

A key point of the invention is that the process of the invention for producing urea is designed such that process streams of the ammonia synthesis can be utilized for the feedstocks, and, in particular, the hydrogen contained in the syngas, generated for example in the steam reforming, can be made utilizable without losses, with only minimal intervention in the ammonia process required.

Concerning the solution proposed here, it should be emphasized that the reactants/sources of the three-stage urea synthesis, namely CO₂, H₂ and NH₃, are already available in the conventional process design of an ammonia plant and that only minor interventions in the ammonia synthesis are necessary. In contrast to this, a conventional formamide synthesis based on CO would entail a complete replanning of the ammonia process. Another important point is the fact that the hydrogen is not lost during the formamide synthesis and can be made available again for the ammonia synthesis after the urea synthesis, since the hydrogen is released again in the reaction of formamide or of formamide and ammonia to form urea.

Further embodiments of the invention are apparent from the dependent claims. The invention is elucidated in detail in the text below.

The process of the invention for producing urea comprises:

a) the preparation of formamide on the basis of carbon dioxide, hydrogen and ammonia, with formation of methyl formate or ammonium formate as intermediate in a catalytic reaction, and
b) the preparation of urea by reaction of the resultant formamide or the resultant formamide with ammonia in the presence of a catalyst,
where the source of carbon dioxide and optionally ammonia is a liquid which is laden with chemically and/or physically bound carbon dioxide and is selected from a methanol phase or an aqueous ammonia solution, and which is obtained directly or indirectly by gas scrubbing of a syngas for the removal of CO₂ using the scrubbing fluid.

The process of the invention is especially suitable for the industrial production of urea. The process of the invention is preferably a continuous process.

Feedstocks/sources used for the process of the invention are, in particular, carbon dioxide, hydrogen and ammonia, which come preferably from a process for ammonia synthesis.

The source used for carbon dioxide and optionally ammonia is a scrubbing fluid which is laden with chemically and/or physically bound carbon dioxide, and which is obtained by gas scrubbing of a syngas for the removal of CO₂ using the scrubbing fluid.

Serving as syngas is preferably a syngas obtained from the steam reforming of gaseous and/or liquid hydrocarbons and subsequent water-gas shift reaction, this syngas being of the type used, for example, for ammonia synthesis. The syngas is preferably a syngas which is generated for the ammonia synthesis. Up until the CO₂ scrubbing, the operation to form the syngas is preferably identical to the conventional ammonia synthesis, preferably by way of steam reforming, including secondary reformer, and subsequent water-gas shift reaction, which is generally performed in two stages as high-temperature CO shift and low-temperature CO shift.

In detail, in the steam reforming, a carbonaceous material, such as natural gas or another hydrocarbon, for example, with optional prepurification, is reacted in a primary reformer with steam, with the carbonaceous material being largely converted into CO, CO₂ and hydrogen. In a downstream apparatus known as a secondary reformer—which here, if used, is considered to be part of the steam reforming procedure—residual carbonaceous material can be reacted by addition of process air, with the process air also introducing nitrogen. In this way the desired H₂/N₂ ratio can be established as well.

In a subsequent water-gas shift reaction, carbon monoxide and steam are converted into carbon dioxide and hydrogen. The water-gas shift reaction is frequently performed in two stages, as a high-temperature shift stage and a low-temperature shift stage. The principal components in the synthesis gas obtained are hydrogen, nitrogen and carbon dioxide. Possible impurities include carbon monoxide, argon and small amounts of hydrocarbons.

Suitable processes for generating a syngas of this kind are known to a skilled person, and in this regard, for example, full reference may be made to A. Nielsen, I. Dybkjaer, Ammonia—Catalysis and Manufacture, Springer Berlin 1995, chapter 6, pages 202-326; M. Appl, Ammonia. Principles and Industrial Practice, WILEY-VCH Verlag GmbH 1999.

Besides the standard route for the provision of the syngas by way of steam reforming, it is possible, alternatively or additionally to the syngas via steam reforming (as an admixture), to use as syngas a gas selected from a coke oven gas, a blast furnace gas or an offgas from cement works, the gas having been subjected to processing where appropriate. Depending on the origin of the gas, it may be useful to process the gas prior to the gas scrubbing. The optional processing may entail, for example, the removal of one or more disruptive constituents from the gas and/or the raising of the CO₂ content by a water-gas shift reaction. The optionally processed gas, selected from a coke oven gas, a blast furnace gas or an offgas from cement works, is suitable as a CO₂ source and possibly also for the ammonia synthesis after processing and removal of CO₂, CO and other oxygen-containing components.

In the customary ammonia synthesis, this syngas is subjected to gas scrubbing with a scrubbing fluid in order to remove CO₂. The gas stream largely freed from CO₂ is subjected to methanization, in which residual CO and CO₂ in the syngas, which constitute catalyst poisons for the ammonia synthesis, are converted into methane. The gas exiting the methanization contains substantially hydrogen and nitrogen in a ratio of 3:1, and some methane, and may be used for the ammonia synthesis itself, following removal of water still present in said gas.

As already observed above, in the process according to the invention, in relation to the ammonia synthesis, a change in the operating regime is possibly necessary only in the context of the CO₂ scrubbing. Described in the text below are judicious embodiments for the removal of CO₂ from the syngas in order to obtain a CO₂-laden fluid for the process of the invention.

CO₂-Laden Methanol Phase

Scrubbing fluid used for the removal/absorption of CO₂ from the syngas, in one particularly preferred embodiment, is a methanol phase as scrubbing fluid. The methanol phase is preferably methanol. Alternatively the methanol phase may include not only methanol but also a small fraction of impurities, such as water or organic solvent, but preferably in an amount of less than 10 vol %, preferably less than 1 vol %. In the course of the subsequent reactions, impurities may lead to byproducts or to reduced conversions, and for these reasons they ought to be avoided as extensively as possible.

One particularly suitable process is that known as the Rectisol process from the companies Lurgi and Linde, which uses methanol as scrubbing fluid for the gas scrubbing. In order to achieve effective absorption of CO₂ into methanol, cold methanol is used in particular. The methanol or the methanol phase is cooled before the gas scrubbing, for example, to temperatures below 0° C., preferably below −20° C., for example in the range from −20° C. to −50° C., preferably −30° C. to −40° C. This can be done using an NH₃ compression refrigeration machine which reaches temperatures of about −33° C., with this constituting an economic method. It is, however, entirely possible, using suitable facilities, to cool the methanol to even lower temperatures, allowing better results to be achieved, although this must be weighed against the profitability. In the course of the gas scrubbing, the methanol warms up again, to about −20° C., for example, but in general remains below 0° C. and must be cooled again after recycling. The gas scrubbing of the syngas for removing CO₂ from the syngas into the scrubbing fluid (methanol or methanol phase) may be carried out, for example, at a pressure in the range from 20 to 50 bar.

The scrubbing fluid obtained after the gas scrubbing is a methanol phase which is laden with physically absorbed CO₂ and which can be passed without regeneration to that extent into the formamide synthesis described below.

In an alternative embodiment a different scrubbing fluid can be used for removing/absorbing CO₂ from the synthesis gas; in this case, after the gas scrubbing, the CO₂ must be removed from the laden scrubbing fluid by desorption and transferred into a methanol phase by absorption. Any scrubbing fluids known in the prior art for gas scrubbing for the purpose of removing CO₂ from a syngas may be used, apart from a methanol phase and an aqueous ammonia solution, which rationally can be used directly as described.

Examples of customary scrubbing fluids are propylene carbonate (Fluor solvent process from Fluor Daniel), a mixture of dimethyl ethers and polyethylene glycol (Selexol® from Union Carbide), alkanolamines, generally in the form of an aqueous solution, such as monoethanolamine (MEA), diglycolamine (DGA), triethanolamine (TEA) and methyldiethanolamine (MDEA), for example, aqueous ammonia solution and aqueous potassium carbonate solution.

For this alternative embodiment the scrubbing fluid employed is preferably aqueous MDEA, admixed with at least one activator, such as MEA or diethanolamine (DEA), N-methylaminoethanol (monomethyl-MEA) or piperazine, for example. Preference here is given to what is called activated MDEA (aMDEA; this process is now referred to as OASE white), which constitutes aqueous MDEA with an addition of monomethyl-MEA or piperazine and is sold by BASF. Another preferred scrubbing fluid is aqueous potassium carbonate solution (Benfield scrub from UOP).

The scrubbing fluid obtained after the gas scrubbing, on the basis of the scrubbing fluids according to this alternative embodiment, is a scrubbing fluid which is laden with chemically and/or physically bound carbon dioxide and which is not a methanol phase. The CO₂ contained in this fluid is therefore desorbed from the laden scrubbing fluid, and the CO₂ released, preferably to the extent required for the formamide synthesis described below, is absorbed into a methanol phase, to give a CO₂-laden methanol phase.

The desorption of CO₂ may be accomplished by the standard methods, which are based, for example, on an increase in the temperature, a reduction in the pressure and/or a reduced fraction of CO₂ in the gas phase over the solution. Specifically this may be accomplished by complete boiling of the solution, lowering of pressure by expansion or the use of a vacuum pump, and the passing of a stripping gas or stripping vapor through the phase.

The fluid obtained after the desorption and absorption procedures described is a methanol phase which is laden with physically absorbed CO₂ and which substantially corresponds in principle to the above-described methanol phase laden with physically absorbed CO₂ and employable directly, as scrubbing fluid, meaning that the further process stages are identical.

The CO₂-laden methanol phase according to the two above embodiments is subsequently reacted, in variant a1) of step a), as a CO₂-containing stream with a hydrogen-containing stream in the presence of a catalyst to form methyl formate (cf. equation (eq. 4) below) and the resultant methyl formate is reacted with an ammonia-containing stream to form formamide (cf. equation (eq. 5) below). The resultant formamide is then reacted, in step b) with ammonia in the presence of a catalyst to give urea (cf. equation (eq. 6) below). The equations set out in the application do not take the stoichiometry into account.

Equations (4) to (7) below show the synthesis, with Cat. denoting catalyst.

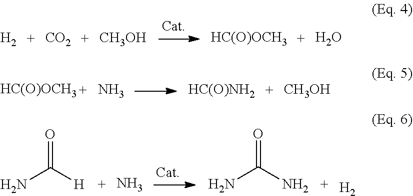

Reaction of CO₂ and H₂ in the methanol phase to form methyl formate as per eq. 4

The reaction as per eq. 4 proceeds in a methanol phase or in a methanol solution. Methanol here acts as solvent and as reactant simultaneously. The reaction of carbon dioxide from the CO₂-containing stream and hydrogen from the hydrogen-containing stream in the methanol phase in the presence of a catalyst forms methyl formate, also referred to as formic acid methyl ester.

The catalytic reaction in eq. 4 and catalysts suitable for this reaction are known to the skilled person. Catalysts known from the literature may be used for this reaction, an example being gold, especially unsupported gold or gold on a support, such as TiO₂ or Al₂O₃, as described in WO 2013/014160 A1, for example; copper catalysts, cobalt catalysts or iridium catalysts, examples being Ir catalysts containing an at least bidentate phosphine ligand, as described in DE 102012019441 A1, for example, or the catalyst [Ir(Cl)(CO)(Ph₃P)₂] described by Vaska et al. in DTIC Document AD-A199 861, 1988. Suitable catalysts are also described in US 2012/071690 A1.

Ruthenium catalysts, such as ruthenium-phosphine complexes, are suitable for example as catalysts for the reaction as per eq. 4. Consequently it is possible, for example, to use a ruthenium catalyst, such as a ruthenium-phosphine complex, as catalyst for the reaction of the CO₂-laden methanol phase and the hydrogen-containing stream to form methyl formate in variant a1). Dimethoxymethane may form as a byproduct, though commonly in small amounts depending on the reaction conditions.

Examples of ruthenium-phosphine complexes as a catalyst for this reaction are described later on below.

In the catalytic reaction of carbon dioxide and hydrogen in the methanol phase to give methyl formate, the catalyst, more particularly the ruthenium-phosphine complex, may be used as a homogeneous catalyst or as an immobilized catalyst. The catalytic reaction with the catalyst, such as with the ruthenium-phosphine complex, may be carried out homogeneously or heterogeneously, with an immobilized catalyst in a fixed bed reactor or with a dissolved catalyst in a fluidized bed reactor, for example.

The catalytic reaction of carbon dioxide and hydrogen in the methanol phase to give methyl formate may be carried out continuously or batchwise, preference being given to continuous operation. The catalytic reaction is carried out preferably in an autoclave or a pressure reactor. An autoclave is suitable for batch operation. A pressure reactor is suitable for continuous operation.

Suitable reaction conditions for the catalysts known for this reaction are known to the skilled person. The text below sets out, in particular, details for suitable reaction conditions when employing ruthenium-phosphine complexes as the catalyst. Where the following paragraphs do not refer expressly to the ruthenium-phosphine complex, they are valid not only for the ruthenium-phosphine complex but also when other suitable catalysts are used.

The concentration of ruthenium-phosphine complex as catalyst in the catalytic reaction of carbon dioxide and hydrogen in the methanol phase to form methyl formate may be situated, for example, in the range from 0.1 mmol % to 5.0 mol %, preferably from 1.0 mmol % to 1.0 mol %, more preferably 2.0 mmol % to 0.1 mol %, based on the molar amount of CO₂.

In addition, the catalytic reaction of carbon dioxide and hydrogen in the methanol phase to give methyl formate, especially when using the ruthenium-phosphine complex as catalyst, takes place preferably in the presence of an acid. This acid serves as a cocatalyst for activating the catalyst and the substrate, and may improve the yield of the reaction.

The acid may be a protic acid (Brønsted acid) or a Lewis acid, with a Lewis acid being preferred. The acid may be an organic acid or an inorganic acid.

A Lewis acid is understood to be an electron pair acceptor, i.e., a molecule or ion with an incomplete noble gas configuration, which is able to accept an electron pair provided by another molecule (Lewis base) and to form a so-called Lewis adduct with said electron pair.

Examples of judicious acids, such as Lewis acids and protic acids, are organoaluminum compounds, such as aluminum triflate (aluminum tris(trifluoromethanesulfonate), Al(OTf)₃ (Tf=−SO₂CF₃)) and aluminum triacetate, organoboron compounds, such as tris(pentafluorophenyl)borane, Bi(OTf)₃, 2,4,6-trimethylbenzoic acid, sulfonic acids, such as p-toluenesulfonic acid, bis(trifluoromethane)sulfonimide (HNTf₂), scandium compounds, such as scandium triflate, perfluorinated copolymers containing at least one sulfo group, as available under the trade name Nafion® NR50, for example, or combinations thereof.

The molar ratio of ruthenium-phosphine complex to acid may lie, for example, in the range from 1:800 to 1:1, preferably from 1:80 to 1:5, more preferably from 1:50 to 1:10.

The catalytic reaction of carbon dioxide and hydrogen in the presence of the catalyst takes place in a methanol phase. The methanol in this case serves simultaneously as reactant and as solvent. The methanol phase is preferably methanol. The methanol phase may alternatively include not only methanol but also a small fraction of impurities, such as water or organic solvents, but preferably in an amount of less than 10 vol %, preferably less than 1 vol %.

In the methanol phase, the ruthenium-phosphine complex is preferably present at least partly in solution. The catalytic reaction of carbon dioxide and hydrogen in methanol is preferably a homogeneous catalytic reaction. The homogeneous catalysis may enable milder reaction conditions and higher selectivities.

Methanol is used at a very high excess, as it is also used as solvent. A stoichiometric excess of H₂ over CO₂ is preferred. For example, for the catalytic reaction, the ratio of p(CO₂):p(H₂) may judiciously be in the range from 2:1 to 1:10, preferably in the range from 1:1 to 1:5, more preferably 1:1.1 to 1:3, where p is the partial pressure of the respective reactant at 23° C. Specific examples of suitable ratios of p(CO₂):p(H₂) are for example 1:2 or 2:3.

The catalytic reaction of carbon dioxide and hydrogen in the methanol phase, particularly when using the ruthenium-phosphine complex as catalyst, takes place preferably at a temperature in the range from room temperature (e.g., 20°) to 150° C. and more preferably in the range from 60 to 140° C. or from 80 to 120° C., with the best results being achieved at about 100° C.

The catalytic reaction of carbon dioxide and hydrogen in the methanol phase, especially when using the ruthenium-phosphine complex as catalyst, takes place preferably at a pressure in the range from 40 bar to 220 bar, more preferably in the range from 80 bar to 200 bar, with the best results being achieved at about 100 to 180 bar.

The appropriate reaction time for the catalytic reaction of carbon dioxide and hydrogen in the methanol phase may vary depending on the other reaction parameters. Judiciously the reaction time is situated for example in a range from 3 to 20 hours, preferably 12 to 18 hours.

In the case of the reaction of carbon dioxide and hydrogen in a methanol phase in the presence of a catalyst, such as a ruthenium-phosphine complex as catalyst, and optionally of an acid, to form methyl formate and water, the product obtained in particular is a methyl formate-containing reaction mixture which is usually drained of unreacted H₂ and CO₂ and then used for the ammonolysis to form the formamide as per eq. 5.

For the expulsion of unreacted H₂ and CO₂, first in particular the pressure and optionally the temperature are lowered, in order to remove the gaseous components (H₂ and CO₂) as extensively as possible from the methyl formate-containing reaction mixture, and then ammonia is added. The pressure at which the gaseous components are isolated is dependent on the prevailing pressures of the coupled process stages.

Depending on the ratio of H₂ to CO₂ that is used, a gradual lowering of pressure or expansion may be worthwhile. In the case of the first expansion, primarily H₂ or H₂/N₂ is separated off, with some CO₂ being likewise removed. This stream can be recycled. The last expansion stage, assisted where appropriate by stripping with stripping gas, is suitable more for combustion in the primary reformer or disposal. The advantage of a gradual expansion is that the gases separated off are still under pressure and so are more suitable for recycling.

The methyl formate-containing reaction mixture from which unreacted H₂ and CO₂ have been removed may be employed for the ammonolysis according to eq. 5 via the following two process regimes, for example. The resultant methyl formate can be removed from the reaction mixture and reacted with ammonia in a separate reaction. The removal may be accomplished simply, for example, by distillation, in which case the methyl formate is the component having the lowest boiling point. In an alternative embodiment, the resultant methyl formate may be reacted with ammonia directly, without prior removal from the reaction mixture. In this case the ammonia is simply introduced into the reaction mixture containing the resultant methyl formate, following catalytic reaction and following expulsion of the unreacted CO₂ and H₂ reactants.

The methyl formate formed in the catalytic reaction of carbon dioxide and hydrogen in methanol is subjected subsequently to an ammonolysis with ammonia to form the formamide. The observations below regarding the reaction of the methyl formate with ammonia are valid irrespectively of whether this is carried out using the methyl formate-containing reaction mixture or using the methyl formate removed from the reaction mixture, as described above.

Reaction of Methyl Formate and NH₃ to Form Formamide as Per Eq. 5

After the formation of methyl formate according to eq. 4, it is subjected to ammonolysis by reaction with ammonia to form formamide, as set out in eq. 5. In this case there is a nucleophilic substitution and methanol is released. The methanol released is recycled, and so formamide constitutes the predominant reaction product. As stated, dimethoxymethane may be formed as a byproduct, though usually in small amounts.

The reaction according to eq. 5 is common knowledge. The reaction conditions are well known to the skilled person. The reaction of the methyl formate with ammonia to give formamide is generally quantitative, and is well known to the skilled person. It is likewise performed in the conventional formamide synthesis starting from CO and MeOH. The reaction of the methyl formate with ammonia to give formamide does not require any catalyst, although the use of a catalyst is not excluded.

The reaction of the methyl formate with ammonia to give formamide and methanol may be carried out, for example, at a temperature in the range from room temperature (e.g. 20° C.) to 100° C., more preferably at 60 to 80° C. The reaction may be carried out, for example, at a pressure in the range from 1 bar (or atmospheric pressure) to 70 bar, preferably in the range from 1 bar (or atmospheric pressure) to 45 bar.

After the reaction, the methanol and any reactants are removed by distillation. Formamide is left as the residue.

The methanol formed in the reaction of methyl formate with the ammonia-containing stream to form formamide may be used preferably for the scrubbing fluid or the methanol-containing fluid in the process again.

Reaction of Formamide and Ammonia to Form Urea as Per Eq. 6 or Reaction of Formamide to Form Urea

The following reaction of the resultant formamide with ammonia according to eq. 6 in the presence of a catalyst leads ultimately to the formation of urea and hydrogen. The possibility of reusing the hydrogen is a particular advantage of the process of the invention. The hydrogen released in the reaction is obtained at the pressure employed for the reaction. It may be returned to the formamide synthesis or to the ammonia synthesis.

In an alternative embodiment, the resultant formamide may be reacted in the presence of a catalyst to form urea and hydrogen, even without addition of ammonia. In this case as well, reusable hydrogen is formed.

Correspondingly, the catalytic synthesis of urea preferably embraces the reaction of formamide with ammonia in the presence of a catalyst, such as a ruthenium-phosphine complex, to form urea and hydrogen. Alternatively the catalytic synthesis of urea embraces the reaction of formamide in the presence of a catalyst, such as a ruthenium-phosphine complex, to form urea and hydrogen, with CO as well being formed in the case of this alternative. In the alternative variant, only formamide is used as starting material for the catalytic synthesis/reaction in the presence of a catalyst to form urea; in particular, no NH₃ is added to the reaction mixture. Starting materials used for the synthesis are therefore formamide or, preferably, formamide and ammonia.

Unless indicated otherwise, the elucidations relating to the catalytic synthesis refer both to the preferred variant and to the alternative variant, as have been indicated above. It will be appreciated that details relating to the added ammonia relate only to the preferred variant.

If hydrogen is to be returned to the formamide synthesis, the pressure of the urea synthesis must be slightly above the pressure for the formamide synthesis, in this case for the reaction according to eq. 4 (see above). In that case the production duo formamide-urea, is in a steady state relative to the self-supplied reactant: hydrogen, which is released from the urea synthesis according to eq. 6 or from the alternative synthesis without addition of ammonia, is used for the synthesis of formamide according to eq. 4/5. Ammonia syngas (N₂/H₂) is needed only at startup or to compensate for H₂ losses.

If the hydrogen is returned to the ammonia synthesis, the hydrogen may be introduced directly into the high-pressure loop of the synthesis or at any desired suction stages of the syngas compressor with or without N₂. If necessary, the pressure of the urea synthesis is adapted accordingly. The suction stages of the compressor (syngas) may have pressures for example of 32, 66, 109 and 195 bar.

Suitable catalysts can be used as catalyst for the reaction of the resultant formamide with ammonia to form urea according to eq. 6, or for the reaction of the resultant formamide to form urea. One preferred embodiment uses a ruthenium catalyst, more particularly a ruthenium-phosphine complex, as catalyst for the reaction of the resultant formamide or of the resultant formamide with ammonia to form urea.

Examples of particularly suitable ruthenium-phosphine complexes as catalyst for this reaction are described later on below.

The text below gives details in particular of suitable reaction conditions when using ruthenium-phosphine complexes as catalyst. If the paragraphs which follow do not relate expressly to the ruthenium-phosphine complex, they are valid not only for the ruthenium-phosphine complex but also when using other suitable catalysts.

The catalyst, more particularly a ruthenium-phosphine complex, may be used as a homogeneous catalyst or as an immobilized catalyst in the catalytic reaction of formamide or of formamide and ammonia to give urea. Two-phase systems with phase transfer catalysis are also possible. The catalytic reaction with the catalyst, more particularly with the ruthenium-phosphine complex, may be carried out homogeneously or heterogeneously, with, for example, an immobilized catalyst in a fixed bed reactor or a dissolved catalyst in a fluidized bed reactor.

The catalytic reaction of formamide or of formamide and ammonia may be carried out continuously or batchwise, with continuous operation being preferred. The catalytic reaction is carried out preferably in an autoclave or a pressure reactor. An autoclave is suitable for batch operation. A pressure reactor is suitable for continuous operation.

The catalytic reaction of formamide or preferably of formamide with ammonia may optionally be carried out, additionally, in the presence of an acid as cocatalyst, and the acid in question may be a Brønsted acid or a Lewis acid. The acid may be an organic acid or an inorganic acid. This acid may lead to the additional activation of the catalyst and/or the formamide, and may improve the yield of the reaction.

Examples of judicious Brønsted acids or Lewis acids are organoaluminum compounds, such as aluminum triflate (aluminum tris(trifluoromethanesulfonate)) and aluminum triacetate, organoboron compounds, such as tris(pentafluorophenyl)borane, sulfonic acids, such as p-toluenesulfonic acid, bis(trifluoromethane)sulfonimide (HNTf₂), scandium compounds, such as scandium triflate, perfluorinated copolymers containing at least one sulfo group, of the kind obtainable under the trade name Nafion® NR50, for example, or combinations thereof.

The catalytic reaction of formamide and ammonia to give urea or the catalytic reaction of formamide to give urea takes place for example at a temperature in the range from 50 to 250° C., preferably in the range from 120 to 200° C., more preferably in the range from 140 to 170° C.

The catalytic reaction of formamide or of formamide with ammonia to give urea takes place for example at a pressure (reaction pressure) in the range from ambient pressure to 150 bar, preferably in the range from 2 bar to 60 bar, more preferably in the range from 5 to 40 bar. In the case of the preferred variant, the reaction may take place optionally under conditions in which liquid or supercritical ammonia is present (critical pressure (NH₃)=113 bar; critical temperature (NH₃)=132.5° C.), which can act as solvent.

In the preferred variant, the amount of ammonia used in the reaction, in equivalents (eq) based on formamide, may be for example in the range from 1 to 300 eq, preferably from 4 eq to 100 eq, more preferably from 29 to 59 eq.

In one preferred embodiment the reaction takes place with about 29 to 59 eq of ammonia, based on formamide, at a pressure in the range from 5 to 40 bar, more particularly 10 to 30 bar. Solvents employed with particular preference in this case are dioxane, more particularly 1,4-dioxane, or toluene.

The reaction preferably takes place, accordingly, with a high stoichiometric excess of ammonia. This enables an improvement in the yield of urea.

The suitable reaction time for the catalytic reaction of formamide or of formamide with ammonia may vary depending on the other reaction parameters. The reaction time of the reaction is situated judiciously, for example, in a range from 1 minute to 24 hours or 30 minutes to 24 hours, preferably 3 to 15 hours, more preferably 6 to 10 hours.

In the process of the invention, the reaction of formamide or, preferably, of formamide with ammonia may be carried out in the absence or presence of solvent, more particularly organic solvent. In the absence of solvent, an optional excess of ammonia in the form of liquid or preferably supercritical ammonia may act as solvent.

In one preferred embodiment the reaction is carried out in a solvent, more particularly an organic solvent. One solvent or a mixture of two or more solvents may be employed, with preference being given to the use of one solvent.

The solvent is preferably an organic solvent, more particularly an aprotic organic solvent. The solvent may be polar or nonpolar, with nonpolar organic solvents being preferred. The solvent is preferably selected such that the catalyst used, preferably the ruthenium-phosphine complex, can be at least partly dissolved therein.

The solvent is preferably selected from the group consisting of cyclic and noncyclic ethers, substituted and unsubstituted aromatics, alkanes and halogenated hydrocarbons, such as di- and trichloromethane, for example, with the solvent being selected preferably from halogenated hydrocarbons, cyclic ethers and substituted or unsubstituted aromatics, preferably from cyclic ethers and substituted or unsubstituted aromatics. Examples of aromatics are benzene or benzene having one or more aromatic substituents (e.g. phenyl) and/or aliphatic substituents (e.g. C₁-C₄ alkyl). Particularly preferred solvents are dioxane, more particularly 1,4-dioxane, toluene, and tetrahydrofuran (THF). However, dichloromethane or trichloromethane may also be used with advantage.

As solvents it is optionally possible alternatively to use ionic liquids as well. Ionic liquids are known to the skilled person. These are salts which are liquid at low temperatures, such as at temperatures of not more than 100° C. The cation of the ionic liquid is selected, for example, from imidazolium, pyridinium, pyrrolidinium, guanidinium, uronium, thiouronium, piperidinium, morpholinium, ammonium and phosphonium, and this cation may be substituted preferably by one or more alkyl groups. The anion of the ionic liquid is selected, for example, from halides, tetrafluoroborates, trifluoroacetates, triflates, hexafluorophosphates, phosphinates, tosylates or organic ions, such as imides or amides, for example.

The catalyst, more particularly the ruthenium-phosphine complex, is present preferably at least partly or completely in solution in the solvent. The catalytic reaction of formamide or of formamide with ammonia to give urea is preferably a homogeneous catalytic reaction. Catalyst and reactants here are present in solution, in other words in the same phase. The homogeneous catalysis may enable milder reaction conditions and possibly higher selectivities and higher turnover numbers (TON) and/or turnover frequency (TOF).

Where a solvent or a solvent mixture is used, the concentration of the one or more solvents is situated, for example, in a range from 5 to 500 mL, preferably from 10 to 300 mL, more preferably from 50 to 250 mL, per 1 mmol of Ru-phosphine complex.

The concentration of catalyst, more particularly of ruthenium-phosphine complex as catalyst, in the reaction may be situated, for example, in the range from 0.05 mol % to 10 mol %, preferably from 0.25 mol % to 5 mol %, more preferably 0.5 mol % to 2 mol %, based on the molar amount of formamide.

After the reaction to form urea, the gaseous components are removed. This relates to hydrogen released and any excess ammonia. The pressure in this case is to remain extremely high, and consequently a reduction in pressure should be avoided. The temperature here may be as desired, but high temperatures are preferred so that more H₂/NH₃ is expelled. The excess NH₃, where present, may be separated from the hydrogen in a recovery stage. The pressure of the urea synthesis and therefore the pressure of the hydrogen released are dependent on the intended use of the hydrogen released, as described above. For more effective removal of the gases, it is possible optionally to use nitrogen—from the formamide synthesis, for example—as stripping agent.

In the case of the variant with added ammonia as reactant, the gas mixture from the urea synthesis (H₂/NH₃) is processed in a manner similar to that customary in ammonia synthesis. The NH₃ removed is reused in the urea and/or formamide synthesis, and the hydrogen removed, with or without nitrogen, may be processed to syngas depending on whether nitrogen has been used as stripping agent. The fact that, in the reaction of formamide and ammonia to give urea, hydrogen is released again and can be reused for the ammonia synthesis or formamide synthesis is a particular advantage of the process of the invention.

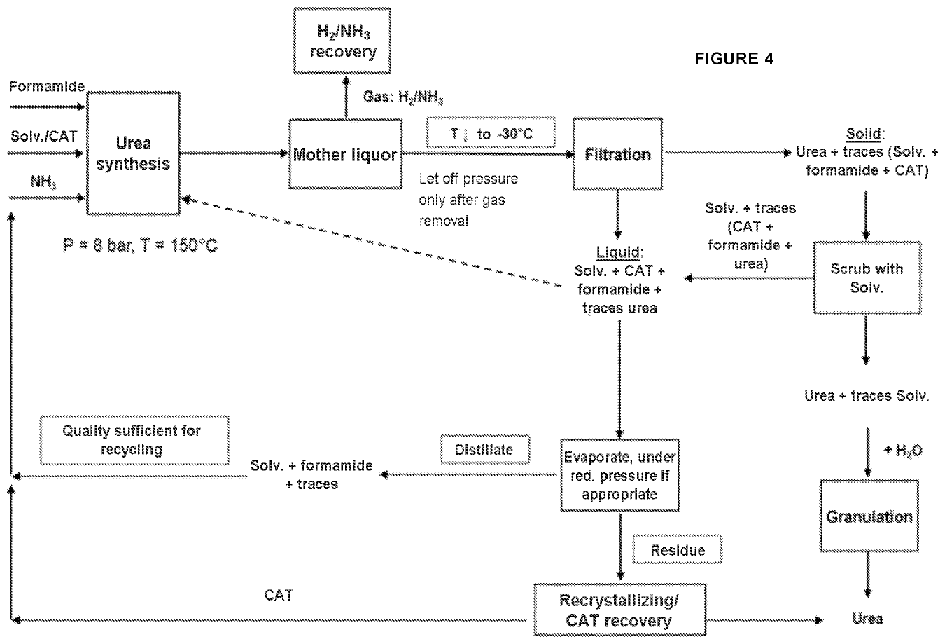

The liquid reaction residue, containing urea, catalyst, any excess formamide and traces of ammonia and also, possibly, solvent, is passed on for processing. Individual possible processing steps are described in more detail below in relation to FIG. 4.

CO₂-Laden Aqueous Ammonia Solution

In a further alternative embodiment in variant a2), the formamide is synthesized not via methyl formate, but instead via ammonium formate as intermediate.

In the case of this alternative embodiment, an aqueous ammonia solution, preferably a dilute aqueous ammonia solution, is used as scrubbing fluid for the gas scrubbing of the syngas for the removal of CO₂, and so CO₂ is bound in the scrubbing fluid at least partly in the form of a corresponding carbamate and carbonate mixture (see equation eq. 7 below). Here it should be pointed out that this is a simplified equation, since CO₂ and NH₃ react with one another and with the water to form a complex mixture of dissolved salts, which in addition to said salts may comprise further compounds. This reaction takes place in the CO₂ scrubbing; in this case it should be emphasized that no regeneration of the scrubbing medium is necessary and the laden scrubbing fluid can be used directly for the further synthesis.

CO₂+H₂O+NH₃→NH₄HCO₃+(NH₄)₂CO₃+H₂NCOONH₄+H₂O (eq. 7)

Of the reaction products according to eq. 7, only ammonium carbamate is unwanted for the further transformations. If the temperature increases (from about 50-60° C. onward), however, ammonium carbamate is hydrolyzed with water to form ammonium carbonate. It is therefore preferable for the ammonia solution to be very highly diluted, since in that case less ammonium carbamate is anticipated.

The aqueous ammonia solution used as scrubbing fluid preferably has an ammonia fraction in the range from 5 to 60 wt %. Preference here is given to using a dilute aqueous ammonia solution, with an ammonia fraction below 30 wt %. In one preferred embodiment the gas scrubbing of the syngas for the removal of CO₂ from the syngas into the scrubbing fluid (aqueous ammonia solution) is carried out at a pressure of 20 to 50 bar and/or at a temperature of below 100° C., preferably in the range from 30 to 70° C.

This scrubbing fluid laden with chemically and physically bound CO₂ is therefore reacted as CO₂-containing stream with a hydrogen-containing stream in the presence of a catalyst and possibly organic solvent or in the presence of a catalyst and an acid, such as a Lewis acid, for example, and possibly organic solvent, to form ammonium formate or to form ammonium formate and formamide (see equation (eq. 8) below). In this case the catalytic reaction of ammonia, carbon dioxide and hydrogen takes place to form ammonium formate or to form formamide and ammonium formate. The ammonium formate formed is converted into formamide by heat treatment (see equation eq. 9 below). Since the reaction is carried out in particular at elevated pressure and temperature, the salts, such as the carbonates and carbamates, decompose in the ammonia solution and release CO₂. The detectable decomposition starts at as low as about 60-70° C.

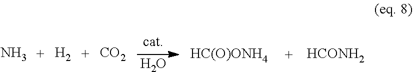

Depending on the operating parameters used, ammonium formate and formamide are produced in different proportions, and under certain reaction conditions, such as a low temperature, it is also possible for only ammonium formate to be formed. When exposed to heat (T), the ammonium formate eliminates water and forms formamide according to the following eq. 9. In principle, therefore, formamide is the only product of the reaction.

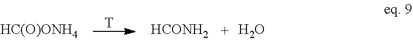

Reaction of CO₂ and H₂ in Aqueous Ammonia Solution to Form Ammonium Formate and Optionally Formamide as Per Eq. 8

The reaction according to eq. 8 proceeds in aqueous ammonia solution. The scrubbing fluid obtained, which is laden with CO₂ and NH₃ at least partly in the form of the carbonates, is reacted with a hydrogen-containing stream in the presence of a catalyst and optionally of organic solvent, or in the presence of a catalyst and an acid, more particularly a Lewis acid, and optionally of organic solvent. The reaction forms ammonium formate and optionally formamide.

As catalyst for the reaction according to eq. 8, suitable catalysts and catalyst types may be used, having already been recited above. One preferred embodiment uses a ruthenium catalyst, more particularly a ruthenium-phosphine complex, as catalyst for the reaction of NH₃, CO₂ and H₂ to give ammonium formate and optionally formamide. Especially when a ruthenium-phosphine complex is employed as catalyst, it is preferred for an acid, more particularly a Lewis acid, to be used as cocatalyst in combination.

Examples of particularly suitable ruthenium-phosphine complexes as catalyst for this reaction are described later on below.

In the text below, in particular, details are given of suitable reaction conditions when employing ruthenium-phosphine complexes as catalyst. Insofar as the following paragraphs do not refer expressly to the ruthenium-phosphine complex, they are valid not only for the ruthenium-phosphine complex but also when other suitable catalysts are used.

The catalyst, e.g., the ruthenium-phosphine complex, may be used as a homogeneous catalyst or as an immobilized catalyst for the catalytic reaction of ammonia, carbon dioxide and hydrogen to give ammonium formate or to give formamide and ammonium formate. Two-phase systems with phase transfer catalysis are also possible. The catalytic reaction with the catalyst, e.g., the ruthenium-phosphine complex, may be carried out homogeneously or heterogeneously, using, for example, an immobilized catalyst in a fixed bed reactor, or a dissolved catalyst in a fluidized bed reactor.

The catalytic reaction of ammonia, carbon dioxide and hydrogen to give ammonium formate or to give formamide and ammonium formate may be carried out continuously or batchwise, with continuous operation being preferred. The catalytic reaction is carried out preferably in an autoclave or a pressure reactor. An autoclave is suitable for batch operation. A pressure reactor is suitable for continuous operation.

The concentration of catalyst, such as of a ruthenium-phosphine complex as catalyst, in the reaction may be situated for example in the range from 0.01 mmol % to 1.0 mol %, preferably from 0.1 mmol % to 0.5 mol %, more preferably 1.0 mmol % to 0.1 mol %, based on the molar amount of NH₃.

The catalytic reaction of ammonia, carbon dioxide and hydrogen may be carried out in the presence of the ruthenium-phosphine complex as catalyst, or in the presence of the ruthenium-phosphine complex as catalyst and of an acid as cocatalyst. Catalytic reaction of ammonia, carbon dioxide and hydrogen to give ammonium formate or to give formamide and ammonium formate takes place preferably in the presence of the ruthenium-phosphine complex and of an acid. The acid here serves as a cocatalyst for activating the catalyst and the reactants, and improves the yield of the reaction.

The acid may be a protic acid (Brønsted acid) or a Lewis acid, a Lewis acid being preferred. The acid may be an organic acid or an inorganic acid.

Examples of judicious acids are organoaluminum compounds, such as aluminum triflate (aluminum tris(trifluoromethanesulfonate), Al(OTf)₃ (Tf=—SO₂CF₃)) and aluminum triacetate, organoboron compounds, such as tris(pentafluorophenyl)borane, Bi(OTf)₃, 2,4,6-trimethylbenzoic acid, sulfonic acids, such as p-toluenesulfonic acid, bis(trifluoromethane)sulfonimide (HNTf₂), scandium compounds, such as scandium triflate, perfluorinated copolymers having at least one sulfo group, of the kind obtainable under the trade name Nafion® NR50, for example, or combinations thereof.

The molar ratio of ruthenium-phosphine complex to acid, if used, may be situated for example in the range from 1:800 to 1:1, preferably from 1:80 to 1:5, more preferably from 1:50 to 1:10.

The catalytic reaction of ammonia, carbon dioxide and water takes place in aqueous ammonia solution which as well as water optionally comprises at least one organic solvent. The aqueous ammonia solution is preferably an aqueous or aqueous-organic solution. Ammonia dissolves very well in water. Physically, carbon dioxide dissolves only minimally in water. The two components react with one another and with the water to form a complex mixture of dissolved salts—for example, ammonium carbonate, ammonium hydrogencarbonate, ammonium carbamate, and other compounds.

The aqueous medium, or the aqueous medium with at least one organic solvent, may be water or, preferably, a mixture of water and at least one organic solvent. The organic solvent is preferably selected such that the ruthenium-phosphine complex employed can be at least partly dissolved therein. Examples of suitable organic solvents are cyclic and acyclic ethers, ketones, nitriles, aromatics, alkanes, halogenated hydrocarbons and alcohols. Specific examples are 1,4-dioxane, tetrahydrofuran, acrylonitrile, acetonitrile, acetone and toluene.

The addition of organic solvent raises the solubility of the catalyst and hence the yield of ammonium formate, thereby promoting the formation of formamide. In this case, however, the formation of formamide is not of principal importance, since formamide is formed in particular in the following step. Moreover, the ammonium formate formed in the course of the reaction is located almost exclusively in the aqueous phase, which facilitates the separation of the reaction products if there is also an organic phase present due to the organic solvent (two-phase system).

If at least one organic solvent is used, the ratio of water to organic component can be varied in various ratios. In this case the ratio of water to the at least one organic solvent, based on the volume, is for example 100:1 to 1:100, preferably 1:10 to 5:1, more preferably 1:5 to 3:1, very preferably 1:2 to 2:1, e.g. 1:1.

In one preferred embodiment at least one organic solvent is added to the aqueous ammonia solution, said organic solvent being miscible with water. This organic solvent is preferably a polar and aprotic solvent. The organic solvent is preferably selected from the group consisting of ethers, more particularly cyclic ethers, such as dioxane, more particularly 1,4-dioxane, and tetrahydrofuran, nitriles, such as acrylonitrile and acetonitrile, and ketones, such as acetone, for example.

In an alternative preferred embodiment at least one organic solvent which is not miscible with water is added to the aqueous ammonia solution, and so a two-phase system made up of aqueous phase and organic phase is formed. Nonpolar organic solvents are generally suitable for this purpose. Examples of such organic solvents are aromatics, such as toluene, esters, ketones, and also ionic liquids. Ionic liquids are regarded here as being organic solvents. Where a two-phase system is formed, care should be taken to ensure sufficient mass transfer between the two phases, by means, for example, of vigorous stirring and/or the use of a phase transfer catalyst, such as sodium dodecyl sulfate, for example.

The catalyst, more particularly the ruthenium-phosphine complex, is present preferably at least partly in solution in the aqueous ammonia solution optionally comprising at least one organic solvent. The catalytic reaction of ammonia, carbon dioxide and hydrogen is preferably a homogeneous catalytic reaction. The homogeneous catalysis may enable milder reaction conditions and possibly higher selectivities and also higher turnover numbers (TON) and/or turnover frequency (TOF).

The amount-of-substance ratio of the reactants may be varied within broad ranges. Based on the stoichiometry of the reaction, the amount-of-substance ratio of the reactants NH₃:CO₂:H₂ is 1:1:1. Departing from the stoichiometry, CO₂ and H₂ are used preferably in excess over NH₃, and H₂ in excess over CO₂, in order to increase the yield of ammonium formate and/or formamide. For example, for the reaction of ammonia, carbon dioxide and hydrogen, a ratio of p(NH₃):p(CO₂):p(H₂) in the range of 1:(1 to 10):(1 to 20), preferably in the range of 1:(1.5 to 10):(2 to 20) and more preferably in the range of 1:(2 to 5):(3 to 12) may be judicious, where p is the partial pressure of the respective reactant at room temperature (23° C.). A specific example of a judicious ratio p(NH₃):p(CO₂):p(H₂) is 8:32:80 (i.e. 1:4:10), for example.

The catalytic reaction of ammonia, carbon dioxide and hydrogen takes place preferably at a temperature in the range from 60 to 180° C. and more preferably in the range from 90 to 110° C.

The catalytic reaction of ammonia, carbon dioxide and hydrogen takes place preferably at a pressure in the range from 35 bar to 210 bar, more preferably in the range from 40 bar to 195 bar, very preferably in the range from 80 to 190 bar.

The suitable reaction time for the catalytic reaction of ammonia, carbon dioxide and hydrogen may vary depending on the other reaction parameters. The reaction time for the reaction is situated judiciously, for example, in a range from 1 minute to 24 hours, preferably 30 minutes to 15 hours.

The ammonium formate formed in the catalytic reaction according to eq. 8 is an intermediate whose formation is further promoted by the water. The thermal treatment according to eq. 9 leads to the elimination of water and to the formation of the desired product, formamide. The reaction may take place proportionally at temperatures as low as about 120° C., and so the ammonium formate product may already undergo proportional reaction to the formamide during the reaction according to eq. 8, albeit only to a relatively minor extent. At higher temperatures, conversion is more complete. At lower temperatures, it is also possible for virtually no formamide to be formed, with only ammonium formate being formed, consequently. Water promotes the formation of ammonium formate by continually transporting product away from the organic phase into the aqueous phase, if a two-phase system is being used.

Where the catalytic reaction takes place in water or in a mixture of water and a water-miscible organic solvent, it is common practice to remove the water continually for the thermal treatment of the ammonium formate to form formamide. If a water-immiscible organic phase is present, it is common to provide for the organic phase to be worked up again or separated. Organic phases promote the formation of formamide.

The catalytic reaction preferably takes place in a two-phase system made up of aqueous phase and organic phase. An example of a suitable organic phase is toluene.

The resultant ammonium formate accumulates almost exclusively in the aqueous phase. The formamide as well, which has already been formed, accumulates very largely in the aqueous phase. The aqueous phase can be separated off and used for the thermal decomposition reaction. Before the thermal decomposition reaction is carried out, the aqueous phase may optionally be processed for purification, by extraction or decanting, for example, in order to remove entrained organic solvent.

The aqueous phase is preferably separated off continuously during the catalytic reaction.

Thermal Treatment of Ammonium Formate to Form Formamide as Per Eq. 9

After the formation of ammonium formate according to eq. 8, the ammonium formate is subjected to an increase in temperature, under which it decomposes to form formamide and water (thermal elimination), as depicted in eq. 9, and so formamide therefore represents in principle the only reaction product. This reaction is common knowledge and requires no catalyst.

The thermal decomposition of ammonium formate into formamide and water according to eq. 9 takes place for example at a temperature in the range from 100° C. to 185° C., preferably more than 130° C. to 185° C., and more preferably at 150° C. to 180° C. The thermal decomposition is carried out preferably at ambient pressure.

For the thermal decomposition there are two fundamental process regimes possible, for example. In one embodiment the resultant ammonium formate can be thermally cleaved in a single-stage reaction, without isolation beforehand, with the reaction mixture being concentrated during this reaction or thereafter, by distillative removal of water.

In the alternative and preferred embodiment, the thermal decomposition is preceded by the processing of the reaction mixture obtained from the catalytic reaction, with the purpose of removing disruptive substances, such as catalyst, byproducts and/or solvent. This takes place depending on the nature of the reaction mixture obtained, by way of variants A and B described below, to give an isolated/purified aqueous phase.

If the catalytic reaction is carried out in a two-phase system with aqueous phase and organic phase, the aqueous phase is separated off from the organic phase (variant A). As elucidated above, the separation preferably takes place continuously.

If the catalytic reaction is carried out in water or in a mixture of water and organic solvent (one-phase system), the aqueous reaction mixture may be purified (variant B) by extraction with a water-immiscible organic solvent, toluene for example, with concluding isolation of the organic phase. This intermediate extraction may likewise be carried out continuously to give a purified aqueous phase.

The isolated or purified aqueous phase obtained according to variant A or variant B and comprising the resultant ammonium formate and optionally formamide already formed in the formate synthesis is subjected to the thermal decomposition in order to convert the ammonium formate into formamide. Generally speaking, water is removed from the reaction mixture by distillation, during and/or after the thermal decomposition reaction.

In one preferred embodiment the thermal decomposition of ammonium formate takes place by a reactive distillation. In this case the reaction mixture obtained from process step a), or, preferably, the isolated or purified aqueous phase obtained according to variant A or variant B and comprising the resultant ammonium formate and optionally formamide already formed in the formate synthesis is subjected to a reactive distillation for the thermal decomposition of the ammonium formate. First water is removed by distillation in this case, thereby shifting the equilibrium in favor of formamide. The formamide product can then be rectified, optionally under reduced pressure. It is, however, also conceivable not to distill formamide at all, and to use the residue, after removal of the water, in the urea synthesis without further processing.

Reaction of Formamide and Ammonia to Form Urea as Per Eq. 6 or Reaction of Formamide to Form Urea

The resultant formamide is subsequently reacted according to step b) with ammonia in the presence of a catalyst in order to give urea (cf. equation eq. 6 above). Alternatively the resultant formamide is subsequently reacted according to step b) in the presence of a catalyst in order to give urea (without addition of ammonia). The reaction according to equation eq. 6 or according to the alternative process regime, and the processing of the reaction mixture obtained, take place in exactly the same way as before in the alternative embodiments on the basis of the CO₂-laden methanol phase, and so in this regard reference may be made to the statements given above.

Ruthenium-Phosphine Complexes as Catalysts

As discussed above, a ruthenium catalyst, more particularly a ruthenium-phosphine complex, is a suitable catalyst for the following reactions performed in the presence of a catalyst:
reaction of formamide with ammonia to form urea and hydrogen (eq. 6) or reaction of formamide to form urea and hydrogen, without addition of ammonia
reaction of the CO₂-laden methanol phase and the hydrogen-containing stream to form methyl formate according to variant a1) (eq. 4)
reaction of the CO₂-laden aqueous ammonia solution and the hydrogen-containing stream to form ammonium formate or to form ammonium formate and formamide according to variant a2) (eq. 8).

The ruthenium-phosphine complex may be used for one, two or all three reactions. It will be appreciated that the ruthenium-phosphine complexes used for the reactions may be the same or different.

The ruthenium-phosphine complex comprises one or more phosphine ligands. The phosphine may be a simple phosphine (monophosphine), a compound having two phosphine groups (diphosphine), a compound having three phosphine groups (triphosphine), or a compound having more than three phosphine groups.

The phosphines are, in particular, trivalent organophosphorus compounds. The phosphine is more particularly a tertiary phosphine or has two, three or more tertiary phosphine groups. The phosphine is, for example, a compound PR¹R²R³, in which R¹, R² and R³ independently of one another each represent an organic radical. The substituents R¹, R² and R³ are preferably independently of one another each substituted or unsubstituted alkyl, substituted or unsubstituted aryl or substituted or unsubstituted heteroaryl.

Identified below are suitable and preferred examples of the groups alkyl, aryl and heteroaryl, and also suitable examples of substituents of corresponding substituted groups, which are valid as examples for all of the references in the present application to these groups or substituted groups, unless explicitly excluded. The examples of the groups alkyl, aryl and heteroaryl are also examples of these groups when they are present as substituents of a group.

Alkyl here also includes cycloalkyl. Examples of alkyl are linear and branched C₁-C₈ alkyl, preferably linear and branched C₁-C₆ alkyl, e.g. methyl, ethyl, n-propyl, isopropyl or butyl and C₃-C₈ cycloalkyl.

Substituted alkyl may have one or more substituents, e.g. halide, such as chloride or fluoride, aryl, heteroaryl, cycloalkyl, alkoxy, e.g. C₁-C₆ alkoxy, preferably C₁-C₄ alkoxy, or aryloxy. Unsubstituted alkyl is preferred.

Examples of aryl are selected from homoaromatic compounds having a molecular weight below 300 g/mol, preferably phenyl, biphenyl, naphthalenyl, anthracenyl and phenanthrenyl.

Examples of heteroaryl are pyridinyl, pyrimidinyl, pyrazinyl, triazolyl, pyridazinyl, 1,3,5-triazinyl, quinolinyl, isoquinolinyl, quinoxalinyl, imidazolyl, pyrazolyl, benzimidazolyl, thiazolyl, oxazolidinyl, pyrrolyl, carbazolyl, indolyl and isoindolyl, where the heteroaryl may be joined to the phosphorus group of the phosphine via any desired atom in the ring of the selected heteroaryl. Preferred examples are pyridinyl, pyrimidinyl, quinolinyl, pyrazolyl, triazolyl, isoquinolinyl, imidazolyl and oxazolidinyl, where the heteroaryl may be joined to the phosphorus group of the phosphine via any desired atom in the ring of the selected heteroaryl.

Substituted aryl and substituted heteroaryl may have one, two or more substituents. Examples of suitable substituents for aryl and heteroaryl are alkyl, preferably C₁-C₄-alkyl, e.g. methyl, ethyl, n-propyl or isopropyl, alkoxy, e.g. methoxy, perfluoroalkyl, e.g. —CF₃, aryl, heteroaryl, cycloalkyl, alkoxy, e.g. C₁-C₆ alkoxy, preferably C₁-C₄ alkoxy, aryloxy, alkenyl, e.g. C₂-C₆ alkenyl, preferably C₃-C₆ alkenyl, silyl, amine and fluorene. Preference is given to unsubstituted aryl, more particularly phenyl, and unsubstituted heteroaryl.

According to one preferred embodiment the phosphine in the ruthenium-phosphine complex is PR¹R²R³, in which R¹, R² and R³ independently of one another are substituted or unsubstituted heteroaryl or substituted or unsubstituted aryl, more particularly phenyl, e.g. tri(heteroaryl)phosphine or tri(aryl)phosphine, or a PR¹R²R³, in which R¹ is alkyl and R² and R³ independently of one another are substituted or unsubstituted heteroaryl and/or substituted or unsubstituted aryl, more particularly phenyl, e.g. di(heteroaryl)alkylphosphine or di(aryl)alkylphosphine.

More preferably the phosphine in the ruthenium-phosphine complex is a compound having two phosphine groups (diphosphine), a compound having three phosphine groups (triphosphine) or a compound having more than three phosphine groups, the phosphine more preferably being a triphosphine. The phosphines having two or more phosphine groups derive preferably from two or more identical or different phosphines PR¹R²R³ as described above, with at least one substituent of the phosphines being linked to one or more other substituents of the phosphines to form a joint group, such as an alkylene group with a valence of two, three or more, as a bridging unit. The details above concerning the substituents and preferred substituents/phosphines are valid analogously for the compounds having more than one phosphine group.

According to one preferred embodiment the ruthenium-phosphine complex contains more than one phosphine group, meaning that there are two or more monophosphines, at least one diphosphine or triphosphine, or a compound having more than three phosphine groups, as ligands in the coordination sphere of the ruthenium.

The bonds between the ruthenium and the phosphine group are formed at least temporarily during the reaction, e.g. a covalent or coordinative bond. It should be noted that in the case of the reaction according to the invention in the presence of the ruthenium-phosphine complex, not all phosphines/phosphine groups in the reaction mixture are necessarily bonded to the ruthenium. In fact the phosphine may be used in excess, meaning that unbonded phosphines/phosphine groups may also be present in the reaction mixture. Particularly if compounds having more than three phosphine groups are used, it is generally the case that not all of the phosphorus atoms are involved catalytically in the reaction; nevertheless, these compounds are also preferred compounds within the present invention.

Particularly preferred are ruthenium-triphosphine complexes where the bridging unit between the phosphorus atoms in the triphosphine is an alkyl or alkylene unit, while the further ligands are heteroaryl with or without substitution or aryl with or without substitution on the phosphorus.

According to one preferred embodiment, the ruthenium-triphosphine complex comprises a triphosphine of the general formula I

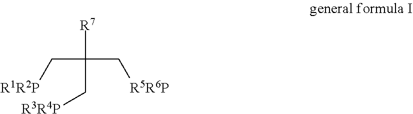

where R¹ to R⁶ independently of one another are substituted or unsubstituted aryl or substituted or unsubstituted heteroaryl, preferably substituted or unsubstituted aryl, and R⁷ is hydrogen or an organic component, preferably alkyl, cycloalkyl or aryl. Examples of suitable substituents for aryl and heteroaryl have been stated above, preference being given to alkyl, more particularly methyl, ethyl, n-propyl, isopropyl, alkoxy, such as methoxy, or perfluoroalkyl, such as —CF₃. The substituted or unsubstituted aryl is preferably unsubstituted aryl, more particularly phenyl. The substituted or unsubstituted heteroaryl is preferably unsubstituted heteroaryl.

The substituents R¹ to R⁶ may be identical or different, and are preferably identical. More preferably R¹ to R⁶ independently of one another are substituted or unsubstituted phenyl. The substituted aryl, more particularly substituted phenyl, may have one, two or more substituents, in ortho- and/or para-position, for example. Examples of suitable substituents have been stated above, preference being given to alkyl, more particularly methyl, ethyl, n-propyl, isopropyl, alkoxy, such as methoxy, or perfluoroalkyl, such as —CF₃. With particular preference R⁷ is alkyl, more preferably methyl or ethyl, more particularly methyl.

One particularly preferred phosphine ligand for the ruthenium-phosphine complex is 1,1,1-tris(diphenylphosphinomethyl)ethane (triphos), which has the following structure:

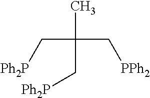

Besides the aforementioned phosphine ligand or ligands, the ruthenium-phosphine complex may have one or more further ligands (nonphosphine ligands), such as, for example, carbenes, amines, amides, phosphites, phosphoamidites, phosphorus-containing ethers or esters, sulfides, trimethylenemethane, cyclopentadienyl, allyl, methylallyl, ethylene, cyclooctadiene, acetylacetonate, acetate, hydride, halide, such as chloride, phenoxide or CO, particularly if the ruthenium-phosphine complex comprises an above-described diphosphine, triphosphine or a compound having more than three phosphine groups.

The one or more further ligands are preferably selected from trimethylenemethane, cyclopentadienyl, allyl, methylallyl, ethylene, cyclooctadiene, acetylacetonate, acetate, hydride, halide, phenoxide, CO or a combination thereof, particular preference being given to trimethylenemethane (tmm). These ligands have a relatively labile bond to ruthenium, and so can easily be substituted by reactant species during the catalytic reaction sequence, with or without assistance from the activator/cocatalyst. Furthermore, a catalyst precursor can be stabilized with these ligands.

In one preferred embodiment the ruthenium-phosphine complex has the following general formula II:

(A)Ru(L)₃ general formula II

in which A is a triphosphine of the general formula I as defined above and L independently of one another in each case are monodentate ligands, it being possible for two monodentate ligands L to be replaced by one bidentate ligand or for three monodentate ligands L to be replaced by one tridentate ligand. Examples of the mono-, bi- or tridentate ligands L are the above-stated further ligands (nonphosphine ligands), in which case they are preferably selected from trimethylenemethane, cyclopentadienyl, allyl, methylallyl, ethylene, cyclooctadiene, acetylacetonate, acetate, hydride, halide, phenoxide, CO or a combination thereof, particular preference being given to trimethylenemethane (tmm). The ligand tmm is a tridentate ligand, for example.

One particularly preferred ruthenium-triphosphine complex has the following structure:

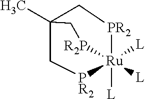

where the substituents R in each case independently of one another are substituted or unsubstituted aryl or substituted or unsubstituted heteroaryl, preferably substituted or unsubstituted aryl, and the substituents L in each case independently of one another are monodentate ligands, it being possible for two monodentate ligands L to be replaced by one bidentate ligand or for three monodentate ligands L to be replaced by one tridentate ligand. Examples of suitable substituents for aryl and heteroaryl have been stated above, preference being given to alkyl, more particularly methyl, ethyl, n-propyl, isopropyl, alkoxy, e.g. methoxy, and perfluoroalkyl, such as —CF₃. The substituted or unsubstituted aryl is preferably unsubstituted aryl, more particularly phenyl. The substituted or unsubstituted heteroaryl is preferably unsubstituted heteroaryl.

The substituents R may be identical or different, and are preferably identical. More preferably R independently at each occurrence is substituted or unsubstituted phenyl. The substituted phenyl may have one, two or more substituents, especially in ortho- and/or para-position. Examples of suitable substituents have been given above, preference being given to alkyl, more particularly methyl, ethyl, n-propyl, isopropyl, alkoxy, e.g. methoxy, and perfluoroalkyl, such as —CF₃. The triphosphine ligand is more preferably triphos.

Examples of the mono-, bi- or tridentate ligands L are the above-stated further ligands (nonphosphine ligands), these ligands being preferably selected from trimethylenemethane, cyclopentadienyl, allyl, methylallyl, ethylene, cyclooctadiene, acetylacetonate, acetate, hydride, halide, phenoxide, CO or a combination thereof, particular preference being given to trimethylenemethane (tmm).

One particularly preferred ruthenium-phosphine complex is [Ru(triphos)(tmm)] with the following structural formula:

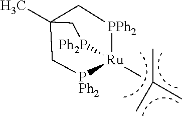

The ruthenium-phosphine complexes identified above are known and may be prepared by the skilled person in accordance with known methods, and/or are available commercially. [Ru(Triphos)(tmm)] is described for example in T. vom Stein et al., ChemCatChem 2013, 5, 439-441.

In the catalytic reaction according to eq. 4, eq. 6 or eq. 8 as described above, independently of one another, the ruthenium-phosphine complex may be used as a homogeneous catalyst or as an immobilized catalyst. The catalytic reaction with the ruthenium-phosphine complex may be carried out homogeneously or heterogeneously, for example with an immobilized catalyst in a fixed bed reactor or with a dissolved catalyst in a fluidized bed reactor.

The catalytic reaction according to eq. 4, eq. 6 or eq. 8 as described above, independently of one another, may be carried out continuously or batchwise, with continuous operation being preferred. The catalytic reaction is carried out preferably in an autoclave or a pressure reactor. An autoclave is suitable for batch operation. A pressure reactor is suitable for continuous operation.

Integration of the Process into an Ammonia Plant

As elucidated above, besides the standard route for the provision of the syngas for the ammonia synthesis by way of steam reforming, it is possible alternatively or additionally (as an admixture to the syngas from steam reforming) to use an optionally processed gas selected from a coke oven gas, a blast furnace gas or a cement works offgas as syngas, preference being given, however, to a syngas for the ammonia synthesis.

As already observed above, in one preferred embodiment the process of the invention is coupled with a customary ammonia synthesis. This ammonia synthesis generally comprises, in this order, the production of a syngas by steam reforming, more particularly comprising the reaction in a primary reformer and a downstream secondary reformer, water-gas shift reaction, in general performed in two stages as high-temperature shift stage and low-temperature shift stage, gas scrubbing of the resultant syngas with a scrubbing fluid for the removal of CO₂, methanization of the purified syngas, and the production of ammonia with the syngas in a conventional way. After the methanization, the syngas is commonly compressed for the subsequent ammonia synthesis.

In the process of the invention for preparing urea, the ammonia synthesis is used as a source of the carbon dioxide, hydrogen and ammonia reactants. As observed above, the CO₂-laden scrubbing fluid obtained from the scrubbing of the syngas is utilized as the source of carbon dioxide.

As a source of hydrogen in the form of the hydrogen-containing stream, it is possible to use a substream of the syngas purified by gas scrubbing (after the gas scrubbing), in which case the substream may be withdrawn prior to the optional methanization of the syngas; a substream of the syngas before the gas scrubbing (crude syngas); a hydrogen obtained from the processing of the products of the urea synthesis; or a combination thereof. As a source of hydrogen it is optionally possible alternatively or additionally to use hydrogen obtained from the processing of the products of the ammonia synthesis.

The withdrawal of the syngas prior to the gas scrubbing, as indicated above, is useful only when the pressure in the formamide synthesis is low, more particularly less than about 35 bar. In other cases, it is necessary to withdraw a highly compressed gas, which has necessarily passed through one or more compressor stages/compression stages. In this case no losses of hydrogen are expected, since the traces of CO/CO₂ in the syngas are reacted in the formamide synthesis. For the removal of the residual CO₂ from the recycle stream from the formamide synthesis, there are three possible solutions (or combination thereof):

1. The hydrogen-containing substream after the gas scrubbing is withdrawn preferably before any optional methanization of the syngas.
2. Return of the offgas from the formamide synthesis to the scrubbing. In this case the operating costs for compression are probably higher, since additional gas stream requires compression.
3. Additional scrubbing, intended to free the offgas from CO₂, using the same scrubbing medium and combining the streams from both scrubs. This stream freed from CO₂ may be processed to the syngas, optionally with the aid of the hydrogen-containing stream from the urea plant.

As described above, the possibility of utilizing the hydrogen generated in the urea synthesis as a source of hydrogen is a particular advantage of the invention. The embodiment wherein a hydrogen-containing substream of the syngas before gas scrubbing is used at least partly for the hydrogen employed relates to a syngas which has not been subjected to the gas scrubbing. The hydrogen-containing substream of the syngas prior to gas scrubbing is a crude syngas also containing CO and CO₂. When it is used, a portion of the crude syngas does not require purification, and the load on the CO₂ scrubbing or CO₂ binding can be reduced by this amount. The preconditions for this have been discussed above. For withdrawal without scrubbing and subsequent compression to be possible, the pressure of the formamide synthesis is to be below 35 bar.

The ammonia obtained from the ammonia synthesis is used as the source of ammonia in the form of the ammonia-containing stream and/or the aqueous ammonia solution. As a source of ammonia it is optionally possible additionally to use ammonia obtained from the processing of the products of the urea synthesis, or else external sources, from a further ammonia plant, for example.

The minor alterations to the conventional ammonia operating regime that are needed for the catalytic urea synthesis of the invention are elucidated in more detail below.

Up to the CO₂ scrubbing, the operation proceeds identically to the conventional ammonia synthesis. The operating regime may change only at the CO₂ scrubbing stage. The methanol-based scrubbing fluid laden with physically absorbed CO₂ (according to the Rectisol process, for example) or the scrubbing fluid laden with chemically absorbed CO₂ and based on aqueous ammonia is passed to the formamide synthesis, without being regenerated, to the extent which is necessary for the urea synthesis. In the case of methanol, the solvent is regenerated during the reaction. MeOH in excess and released during the reaction (eq. 4 and eq. 5) is processed and returned. In the case of aqueous ammonia, the laden solution is at least very largely consumed.

The syngas purified by gas scrubbing and/or the crude syngas can be subdivided, and a substream thereof diverted and passed to the formamide synthesis as a source of hydrogen (see the relevant remarks above). Alternatively or additionally, the hydrogen formed in the urea synthesis may be utilized as a source of hydrogen. Particular preference is given to utilizing the hydrogen formed in the urea synthesis, which according to the stoichiometry produces sufficient hydrogen for the formamide synthesis; additionally, a substream of the purified syngas and/or of the syngas may be utilized, provided the urea synthesis is not running or is not running in steady state, and in order to compensate possibly production-related hydrogen losses in the urea synthesis. The division of the purified syngas may take place, for example, before the methanization or after the compression of the syngas, depending on the parameters selected in the formamide synthesis. The highly hydrogen-containing gas from the H₂ recovery after the ammonia synthesis may likewise be passed into the formamide synthesis. Synthesized ammonia is subsequently passed into the formamide synthesis for the reaction (2) or utilized, optionally, for the production of aqueous ammonia, but with the same purpose.

The “product streams” of the formamide synthesis, in the case of the variant based on a methanol phase, include not only formamide itself but also the water, nitrogen from the syngas, and methanol. The water is separated off and passed to a water processing facility or disposed of.

After reprocessing, the methanol may be reused, for example, for scrubbing (Rectisol).

The processing of the methanol is necessary in order to free it from water, since the water is also present in the crude syngas and is entrained. Moreover, water is formed in the reaction. The reprocessing of the methanol to remove water is necessary because the water is a disrupter when the methanol is reused in scrubbing. At high water content, a water/methanol mixture may even freeze, depending on the temperature employed. The nitrogen can be combined, for example, with the water from the urea synthesis, and processed to the syngas. This syngas fraction corresponds in principle to the quantity withdrawn from the syngas for the formamide synthesis. This fraction of the syngas, with or without additional processing, can be combined with the rest of the syngas prior to the methanization, since this gas may come in at a lower pressure and has to be compressed. The precise position of the methanization and of the reintroduction of the portion of the syngas is dependent on the pressure of the formamide synthesis and of the urea synthesis, which theoretically is arbitrary. Reference is made to the observations above concerning the withdrawal of the syngas.

Below, the invention is described with reference to exemplary embodiments, which are elucidated in more detail with reference to the figures. The specific exemplary embodiments are not intended in any way to limit the scope of the claimed invention. In these figures:

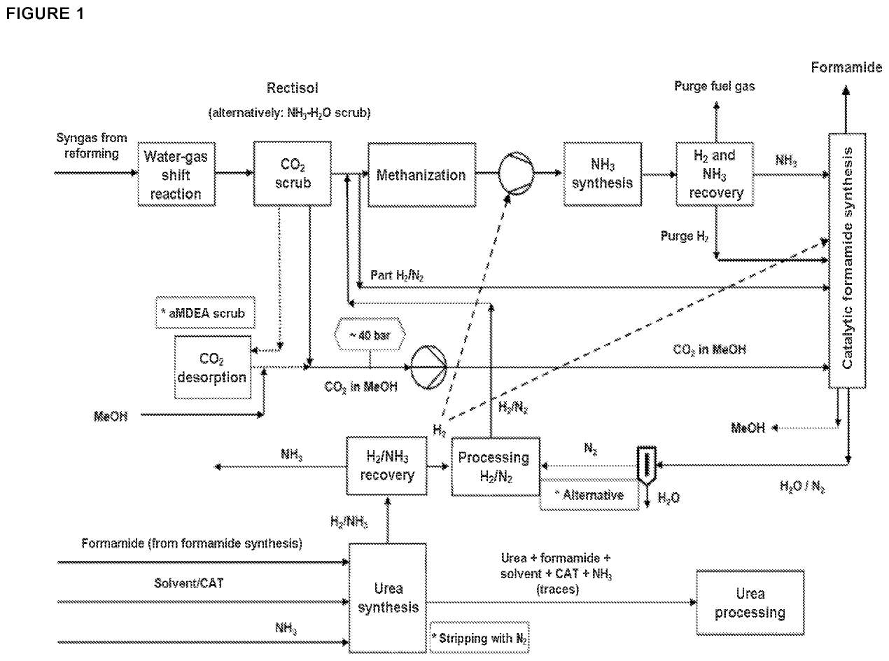

FIG. 1 shows a block diagram of a specific example of a process of the invention for producing urea, coupled with an ammonia synthesis;

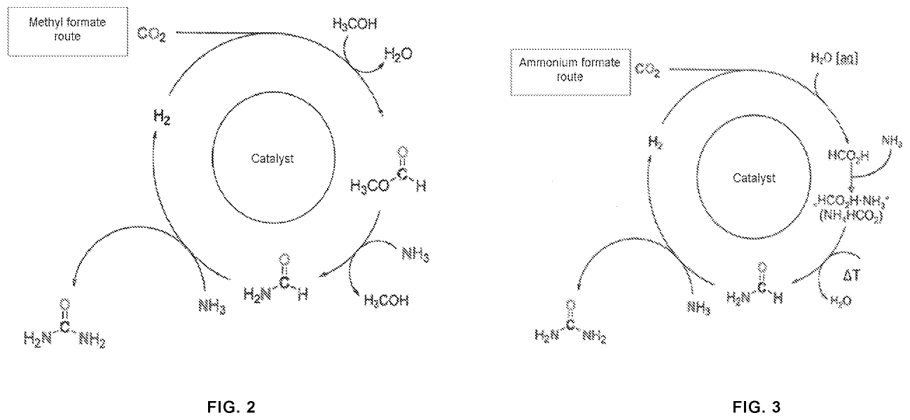

FIG. 2 shows a reaction scheme of the variant of the formamide synthesis via the methanol route (methyl formate);

FIG. 3 shows a reaction scheme of the variant of the formamide synthesis via the aqueous route (ammonium formate);

FIG. 4 shows a block diagram of an example of the processing of the urea.

FIG. 1 shows a block diagram of an example of a process of the invention for producing urea, which is coupled with an ammonia synthesis. A syngas, which in this case comes from steam reforming and is intended for the ammonia synthesis, comprises hydrogen, nitrogen, and carbon monoxide. The syngas is subjected to a water-gas shift reaction (water-gas conversion reaction) in order to convert carbon monoxide into carbon dioxide. After that a gas scrub is performed in order to remove carbon dioxide from the syngas. The gas is scrubbed by means of a scrubbing fluid, with the scrubbing fluid becoming laden with chemically and/or physically bound carbon dioxide during the gas scrubbing.

In this exemplary embodiment, the gas scrubbing takes place with a scrubbing fluid which is methanol (Rectisol). During the gas scrubbing, the methanol becomes laden with carbon dioxide. The resultant stream is used as a CO₂-containing stream for the catalytic formamide synthesis (methyl formate intermediate). The catalytic formamide synthesis is shown only schematically in FIG. 1; as described, it may be carried out in one stage or two stages (with or without isolation of the intermediate stage) to form methyl formate as intermediate and then to form formamide as the end product. A reaction scheme for this variant is shown in FIG. 2. The resultant formamide is passed as a reactant to the urea synthesis.

An alternative embodiment is represented with dashed lines. In this case the gas is scrubbed with an aqueous solution of methyldiethanolamine with piperazine as scrubbing fluid (aMDEA scrubbing). Alternatively, any conventional scrubbing fluid known to the skilled person may be used, an example being aqueous potassium carbonate (Benfield scrubbing). CO₂ is desorbed by CO₂ desorption from the carbon dioxide-laden scrubbing fluid, and the CO₂ released is absorbed into methanol, to give a CO₂-laden methanol phase. In analogy to the Rectisol scrubbing described above, the resulting stream is used as a CO₂-containing stream for the catalytic formamide synthesis, for which it is necessary to increase the pressure of the CO₂ before or after the absorption into MeOH.

In a further, alternative embodiment, not shown, the gas is scrubbed with an aqueous ammonia solution (NH₃—H₂O scrubbing). In this case, CO₂ is bound physically and in the form of carbonates and carbamate in the scrubbing fluid and is used directly as a CO₂-containing stream for the catalytic formamide synthesis (ammonium formate intermediate). In the case of this variant, the catalytic formamide synthesis may be carried out as described in one stage or two stages (with or without isolation of the intermediate) to form ammonium formate as intermediate, and subsequently to form the formamide as the end product. A reaction scheme for the alternative variant based on the aqueous ammonia solution with the subsequent urea synthesis is shown in FIG. 3.

As a hydrogen-containing stream, a substream of the syngas, containing hydrogen and nitrogen, can be diverted before or after the gas scrubbing and used for the formation of formamide (in FIG. 1, after the gas scrubbing and before the methanization). The rest of the syngas is subjected to methanization, in order to remove further carbon monoxide and/or carbon dioxide from the syngas, by formation of methane. After compression, the syngas is then used for the ammonia synthesis. The reaction mixture recovered from the ammonia synthesis is worked up for recovery of NH₃ and H₂. The resultant ammonia and the resultant hydrogen may also be used for the formamide synthesis. The resultant hydrogen may be returned to the NH₃ synthesis or used as a make-up stream for the formamide synthesis, if small amounts of H₂ are lost in the formamide synthesis. Other variants of the syngas withdrawal have been described above.

As elucidated above, the hydrogen from the urea synthesis is preferably used as a hydrogen-containing stream for the formation of formamide (shown as a dashed arrow in FIG. 1), and the substream of the syngas may be utilized in the manner of a supplement if the urea synthesis is not running and/or in order to compensate any hydrogen losses in the urea synthesis.

The methanol likewise formed in the reaction of methyl formate with ammonia to form formamide is reused in the process for the scrubbing fluid or the methanol phase. It is reprocessed together with excess MeOH from the scrubbing fluid, and is returned to the scrubbing.

Specific examples of the reactions are given later on below.

The urea synthesis is followed by the processing of the resultant mixture comprising urea, formamide, solvent, catalyst (CAT), ammonia (NH₃) and hydrogen (H₂). Details of the possible processing of the product obtained in the urea synthesis are elucidated below in FIG. 4. Hydrogen obtained from the processing of the products of the urea synthesis is used for the syngas after H₂ and NH₃ recovery and optional processing of H₂/N₂. In other words, the hydrogen formed in the production of the urea from formamide and ammonia can be returned into the operation. The ammonia obtained in the H₂/NH₃ recovery may likewise be reused at numerous points in the operation.

FIG. 2 shows a schematic reaction scheme of the variant of the formamide synthesis via the “methanol route” to give formamide via methyl formate, and the reaction of the formamide to give urea, corresponding to equations eq. 4, eq. 5 and eq. 6, with the reactant circuit and product circuit. The reactions shown have been described in detail above.

FIG. 3 shows a schematic reaction scheme of the variant of the formamide synthesis via the “aqueous route” to give formamide via ammonium formate, and the reaction of the formamide to give urea, corresponding to equations eq. 8, eq. 9 and eq. 6, with the reactant circuit and product circuit. The reactions shown have been described in detail above.

FIG. 4 shows a block diagram of an example for the possible processing of the product obtained in the urea synthesis. In FIG. 4, illustratively, a pressure of 8 bar and a temperature of 150° C. are specified for the urea synthesis. The temperature may be significantly lower, but the pressure may also be significantly higher. Following production of the urea, the temperature may possibly be lowered (heat integration); otherwise a high-pressure flash is produced. A high temperature is advantageous, though, since more hydrogen and ammonia are separated off, even at high pressure. A change in pressure is to be avoided as far as possible. As described above, hydrogen and ammonia are stripped in gas form from the mother liquor, optionally using nitrogen as the stripping gas. This gas mixture is processed in a manner similar to that customary in the ammonia synthesis. The liquid reaction residue, comprising urea, solvent, catalyst, formamide and traces of ammonia, is processed further in accordance with customary methods. The mother liquor is cooled down to about −30° C. and subjected to filtration. In the course of cooling, the predominant fraction of the urea is precipitated from the mother solution. The residue contains urea and traces of solvent, formamide and catalyst. These traces are removed by washing with fresh, optionally cold solvent, and the residue, containing urea and traces of the solvent, is subjected to granulation in order to obtain the urea.

Alternatively, following removal of urea, the filtrate can be admixed with fresh components NH₃ and formamide (in amounts which have been consumed) and returned directly to the urea synthesis. Ideally, the solvent which remains when the urea isolated by filtration is washed is combined with the filtrate and concentrated, in order to avoid losses of catalyst. The solvent removed by distillation can be reused for the wash, and the concentrated solution can be returned to the urea synthesis.

The processing of the residue (washing with fresh cold solvent, removal of the catalyst, etc.) may take place alternatively in a separate circuit, which is also closed. The components recovered may be admixed to the main streams (e.g. urea, catalyst or formamide; dashed arrow in FIG. 4) at intervals of time. The wash solution used for the washing is combined with the filtrate. The resultant mixture contains solvent, catalyst, formamide and traces of urea. The mixture is concentrated by evaporation, optionally under reduced pressure. The solvent removed by distillation and the concentrated solution may be reused as elucidated above, with this being the most judicious approach. Alternatively, given sufficient quality, formamide, solvent and distillate may be returned to the process. The residue is recrystallized in order to separate urea and catalyst from one another. The catalyst can be reused in the process.

### EXAMPLES

### Synthesis of [Ru(triphos)(tmm)]

A 35 mL Schlenk tube was filled with 319 mg (1.00 mmol) of [Ru(cod)(methylallyl)] (cod=1,5-cyclooctadiene) and 624 mg (1.17 mmol) of 1,1,1-tris(diphenyl-phosphinomethyl)ethane in 20 mL of toluene. The reaction mixture was stirred and was heated at 110° C. for 2 h, cooled to room temperature and concentrated under reduced pressure. Following treatment with 15 mL of pentane, the precipitating complex was isolated, washed with pentane (3×10 mL) and dried under reduced pressure overnight, to give [Ru(triphos)(tmm)] as a pale yellow powder (0.531 g, 0.678 mmol, 68% yield). The identity was confirmed by ¹H, ¹³C APT and ³¹P NMR spectra.

### Examples 1-9

### Synthesis of urea from formamide and ammonia with Ru(triphos)(tmm)

The urea was synthesized in accordance with the following equation:

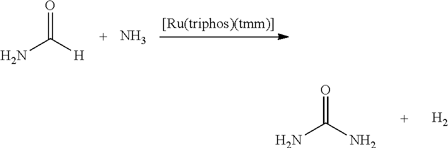

High-pressure batch experiments were performed in a 10 mL autoclave fitted with a glass insert and a magnetic stirring rod. When 2 mL of 1,4-dioxane and 0.6 g of NH₃ were used, the reaction pressure was about 30 bar in the hot state (reaction temperature) and the pressure in the cold state (room temperature) was about 8-10 bar. Before being used, the autoclave was evacuated for at least 30 minutes and filled repeatedly with argon. The catalyst [Ru(triphos)(tmm)] (7.8 mg, 0.01 mmol) was weighed under an argon atmosphere into a Schlenk tube and dissolved in 1,4-dioxane (2.0 mL). Following addition of formamide (40 μL, 1.00 mmol), the reaction mixture was transferred to the autoclave with a canula under an argon countercurrent. Liquid NH₃ (between 0.5 and 1.0 g) was introduced into the autoclave, and the autoclave was sealed. The reaction mixture was stirred and was heated to the respective reaction temperature in an aluminum cone for the respective reaction time. After cooling to room temperature, the autoclave was cautiously let down with air. Following removal of the solvent under reduced pressure, the reaction solution obtained was analyzed by ¹H and ¹³C NMR spectroscopy, using mesitylene as internal standard, and the yield was determined.

The experiment was repeated a number of times, with the catalyst loading, solvent, reaction temperature and reaction time being varied as shown in table 1 below. Table 1 also shows the yield of urea obtained.

The catalyst loading is the amount of catalyst used in mol %, relative to the amount of formamide used (in mol).

TABLE 1
Ru-catalyzed synthesis of urea from formamide and ammonia*

| | Catalyst | | Reaction | Reaction | |
|---|---|---|---|---|---|
| | loading | | temperature | time | Yield |
| Ex. | [mol %] | Solvent | [° C.] | [hours] | [%] |
| 1 | 1.00 | 1,4-Dioxane | 150 | 5 | 44 |
| 2 | 1.00 | 1,4-Dioxane | 150 | 10 | 64 |
| 3 | 1.00 | 1,4-Dioxane | 150 | 15 | 57 |
| 4 | 1.00 | 1,4-Dioxane | 130 | 10 | 12 |
| 5 | 1.00 | 1,4-Dioxane | 110 | 10 | 1 |
| 6 | 0.50 | 1,4-Dioxane | 150 | 10 | 26 |
| 7 | 0.25 | 1,4-Dioxane | 150 | 10 | 14 |
| 8 | 1.00 | Toluene | 150 | 10 | 53 |
| 9 | 1.00 | THF | 150 | 10 | 47 |

*Reaction conditions: [Ru(triphos)(tmm)], 1 mmol formamide, 2 mL solvent, 0.5-1.0 g NH₃

### Example 10

### Preparation of Ru(Triphos)(Tmm) In Situ for Synthesis of Urea

The catalyst Ru(triphos)(tmm) was formed in situ from the catalyst precursor [Ru(cod)(methylallyl)₂] and triphos.

For this, 1 mol % of [Ru(cod)(methylallyl)₂], 1.3 mol % of triphos, 1 mmol of formamide, 2 mL of 1,4-dioxane and 0.6 g of NH₃ were reacted at 150° C. for 10 h. The pressure was about 8 bar in the cold state and about 30 bar at 150° C. The yield of urea was 51%.

### Example 11

### Synthesis of Urea from Formamide in the Absence of Ammonia

1 mol % of [Ru(Triphos)tmm], 1 mmol of formamide and 2 mL of 1,4-dioxane were reacted at 150° C. and 15 bar for 10 h. The yield of urea was 7%.

### Examples 12 to 18

### Catalytic Activity of Ru-Phosphine Complexes as a Function of the Ligands on the Phosphorus

The catalytic activity of various Ru-phosphine complexes in the synthesis of urea from formamide and ammonia was tested as a function of the ligands on the phosphorus. Table 2 indicates the complexes (catalysts) studied, the reaction conditions and the yields obtained. In the experiments the reaction pressure was about 30 bar at the reaction temperature and the pressure in the cold state was about 8 bar, except in ex. 15.

Ruthenium-triphosphine complexes with the following structure were studied:

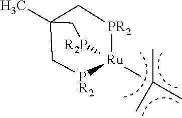

The nature of the substituent R is shown in table 2 below; where not all of the substituents R on the three phosphorus atoms are the same, the substituents R on a first P atom are identified as R¹, on a second P atom as R², and on a third P atom as R³. For example, the complex of ex. 16 has two phenyl groups on two phosphine groups, and the third phosphine group has two isopropyl groups.

The ruthenium-triphosphine complex additionally possesses the tridentate ligand trimethylenemethane.

The pressures reported in the table relate to room temperature (about 23° C.). The autoclave was charged at room temperature and then brought to reaction temperature and reaction pressure.

TABLE 2

| Ex. | R = | Reaction conditions | Urea yield |
|---|---|---|---|
| 12 | 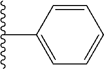 [SMILES: *c1ccccc1] | 1 mol % cat., 1 mmol formamide, 2 mL 1,4- dioxane, 0.6 g NH₃, 150° C., 10 H | 64% |
| 13 | 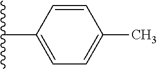 [SMILES: *c1ccc(C)cc1] | 0.5 mol % cat., 1 mmol formamide, 2 mL 1,4- dioxane, 8 bar NH₃, 150° C., 20 h | 8% |
| 14 | 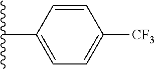 [SMILES: *c1ccc(C(F)(F)F)cc1] | 0.5 mol % cat., 1 mmol formamide, 2 mL 1,4- dioxane, 8 bar NH₃, 150° C., 20 h | 8% |
| 15 | 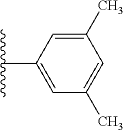 [SMILES: *c1cc(C)cc(C)c1] | 1 mol % cat., 2 mol % B(C₆F₅)₃, 1 mmol formamide, 2 mL 1,4- dioxane, 4 bar NH₃, 150° C., 20 h | 16% |
| 16 | 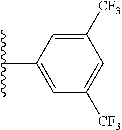 [SMILES: *c1cc(C(F)(F)F)cc(C(F)(F)F)c1] | 0.5 mol % cat., 1 mmol formamide, 2 mL 1,4- dioxane, 8 bar NH₃, 150° C., 20 h | 2% |
| 17 | 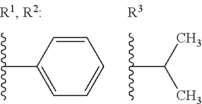 | 1 mol % cat., 1 mmol formamide, 2 mL 1,4- dioxane, 8 bar NH₃, 150° C., 20 h | 30% |
| 18 | 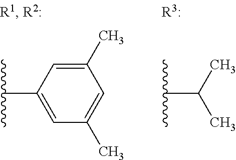 | 0.2 mol % cat., 5 mmol formamide, 4.0 g NH₃, 150° C., 20 h | 6% |

### Examples 19 to 21

### Catalytic Activity of Ru-Phosphine Complexes as a Function of the Additional Ligands on Ruthenium (Nonphosphine Ligands)

The catalytic activity of various Ru-phosphine complexes in the synthesis of urea from formamide and ammonia was tested as a function of the nonphosphine ligands on the ruthenium. Table 3 indicates the complexes (catalysts) studied, the reaction conditions and the yields obtained. In the experiments the pressure was about 30 bar at the reaction temperature and the pressure in the cold state (room temperature) was about 8-10 bar. Example 19 corresponds to example 12.

Ruthenium-triphosphine complexes with the following structure were studied:

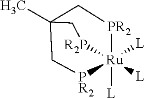

The three ligands L are shown in table 3 below, with one ligand L being designated L¹, a second ligand L L², and a third ligand L L³. In example 19 the three ligands L are formed together by the tridentate ligand trimethylenemethane (tmm). The pressures reported in the table relate to room temperature (about 23° C.). The autoclave was charged at room temperature and then brought to reaction temperature and reaction pressure.

TABLE 3

| Ex. | L = | Reaction conditions | Urea yield |
|---|---|---|---|
| 19 | 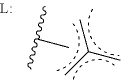 | 1 mol % cat., 1 mmol formamide, 2 mL 1,4-dioxane, 0.6 g NH₃, 150° C., 10 h | 64% |
| 20 | L¹ = CO; L² = H; L³ = Cl | 1 mol % cat., 1 mmol | 18% |
| | | formamide, 2 mL 1,4-dioxane, | |
| | | 8 bar NH₃, 150° C., 20 h | |
| 21 | L¹ = CO; L², L³ = H | 1 mol % cat., 1 mmol | 24% |
| | | formamide, 2 mL 1,4-dioxane, | |
| | | 0.l7 g NH₃, 150° C., 10 h | |

### Examples 22 to 28

### Catalytic Activity of Ru-Phosphine Complexes as a Function of Catalyst Concentration

The catalytic activity as a function of the catalyst concentration was tested for the following reaction conditions:

Catalyst: [Ru(triphos)(tmm)], 1 mmol formamide, 2 mL 1,4-dioxane, 0.6 g NH₃, 150° C., 10 h, with the catalyst concentration being varied. The reaction pressure was about 30 bar at the reaction temperature and the pressure in the cold state was about 8-10 bar.

Table 4 indicates the catalyst concentration (in mol % based on formamide) used under these reaction conditions, and the yields obtained.

TABLE 4

| Ex. | c(cat.) [mol %] | Urea yield [%] |
|---|---|---|
| 22 | 0.05 | <1 |
| 23 | 0.25 | 16 |
| 24 | 1 | 40 |
| 25 | 5 | 63 |

The catalytic activity as a function of the catalyst concentration was additionally tested for the following reaction conditions:

Catalyst: [Ru(triphos)(tmm)], 1 mmol formamide, 2 mL 1,4-dioxane, 4 bar NH₃ at room temperature (around 23° C.), 150° C., 20 h, with the catalyst concentration being varied.

Table 5 indicates the catalyst concentration (in mol % based on formamide) used under these reaction conditions, and the yields obtained.

TABLE 5

| Ex. | c(cat.) [mol %] | Urea yield [%] |
|---|---|---|
| 26 | 0.25 | 25 |
| 27 | 0.5 | 30 |
| 28 | 2 | 33 |

### Examples 29 to 35

### Catalytic Activity of Ru-Phosphine Complexes as a Function of the Solvent Concentration

The catalytic activity as a function of the solvent concentration was tested for the following reaction conditions:

Catalyst: 1 mol % [Ru(triphos)(tmm)], 1 mmol formamide, 0.6 g NH₃, 150° C., 10 h, with the solvent concentration being varied. The reaction pressure was about 30 bar at the reaction temperature and the pressure in the cold state was about 8-10 bar. The solvent was 1,4-dioxane.

Table 6 indicates the amount of 1,4-dioxane used under these reaction conditions, in ml (V(1,4-dioxane) [mL]), and the yields obtained.

TABLE 6

| Ex. | V(1,4-dioxane) [mL] | Urea yield [%] |
|---|---|---|
| 29 | 0.5 | 53 |
| 30 | 0.8 | 45 |
| 31 | 1.1 | 50 |
| 32 | 1.4 | 60 |
| 33 | 1.7 | 42 |
| 34 | 2.3 | 37 |
| 35 | 2.6 | 31 |

### Example 36

A syngas generated for an ammonia synthesis is subjected to gas scrubbing, using an aqueous ammonia solution having an ammonia fraction of about 5 to 60 wt %, preferably about 5 to 30 wt %, for removing the carbon dioxide from the syngas at the syngas generation pressure of around 36 bar and at a temperature of about 40° C. to 60° C., in an absorber. CO₂ removal from crude syngas with aqueous ammonia is a commonplace process and is also described, for example, in US 2018/0282265 A1.

Without further processing, the resulting solution or suspension of chemically and physically bound carbon dioxide is mixed in a separate reactor with a substream of the syngas purified by gas scrubbing or with a substream of the unpurified syngas (crude syngas) as hydrogen source. Mixing takes place so as to maintain, at least approximately, a ratio p(NH₃):p(CO₂):p(H₂) in the mixture of 8:32:80 (p=partial pressure at room temperature (23° C.)).

A catalyst solution composed of the ruthenium-phosphine complex [Ru(Triphos)(tmm)] in solution in toluene at a concentration of 0.7 pmol of catalyst per mL of toluene and 25 equivalents of Al(OTf)₃, based on the amount of substance of the catalyst, or Nafion as acid are added to the mixture. The resultant mixture forms a two-phase system (water/toluene) and is reacted at a temperature of about 100° C. and at a pressure of about 180 bar for 12 h. The TON based on ammonium format is 6941, for example.

Following the reaction, the aqueous phase is isolated. The aqueous phase is then subjected to a thermal decomposition treatment at 176° C. and ambient pressure, which converts the ammonium formate contained therein to an extent of 83% into formamide. Water is removed by distillation during or after the thermal decomposition treatment. The resultant formamide product may be subsequently rectified for purification.

### Example 37

A syngas generated for an ammonia synthesis is subjected to gas scrubbing for removal of CO₂. For this purpose the carbon dioxide is removed from the syngas using what is called a Rectisol process, a physical gas purification process with methanol as scrubbing medium, at the prevailing syngas generation pressure of around 36 bar and at a temperature of about −40° C. to −20° C. For the absorber temperature, a figure of between −40° C. and −30° C. is preferred here. The resulting methanol solution of physically bound carbon dioxide is brought to an increased pressure without further processing, with optional subsequent low-temperature integration and with the aid of a pump. The laden methanol solution is subsequently mixed in a separate reactor with a substream of the syngas purified by gas scrubbing, or with a substream of the unpurified syngas (crude syngas), as a hydrogen source, and admixed with the catalyst in solution in methanol, in order to carry out the reaction sequence according to eq. 4 and eq. 5. To imitate the industrial operation, the ruthenium-phosphine complex [Ru(Triphos)(tmm)] (0.5 μmol, 0.001 mol % based on methanol) and aluminum triflate (Al(OTf)₃ (12.5 μmol, 0.025 mol % based on methanol) as Lewis acid in 2 mL of methanol were admixed with the CO₂ in a 10 mL autoclave at room temperature, and so the pressure of the reaction mixture was around 40 bar (syngas generation pressure). Hydrogen or a hydrogen-nitrogen mixture (molar ratio 3:1, imitating the ammonia syngas) was passed into the autoclave, so that the total pressure in the autoclave was brought to 120 bar. The mixture was reacted at a temperature of 100° C. and at a resultant pressure of about 180 bar for 18 hours, to form a reaction mixture comprising methyl formate. Depending on catalyst loading, a turnover number (TON) of 4000 is attained for methyl formate.

The reaction mixture was cooled, the pressure was lowered, and the gaseous reactants were removed. The mixture comprising methyl formate was admixed with NH₃ (8 bar total pressure at room temperature) and stirred at a temperature of 60° C. (reaction pressure of around 27 bar) for an hour. The methanol released can be subsequently removed by distillation from the formamide product. The reaction of methyl formate with ammonia to give the formamide is virtually quantitative.

1.-20. (canceled)

21. A process for preparing urea comprising:
preparing formamide based on carbon dioxide, hydrogen, and ammonia, forming methyl formate or ammonium formate as an intermediate in a catalytic reaction; and
preparing urea by reacting the formamide or the formamide with ammonia in the presence of a catalyst,
wherein a source of carbon dioxide is a liquid laden with chemically and/or physically bound carbon dioxide and selected from a methanol phase or an aqueous ammonia solution that is obtained by gas scrubbing of a syngas for removal of carbon dioxide using a scrubbing fluid, wherein either:
the scrubbing fluid is a methanol phase, or carbon dioxide is desorbed from the scrubbing fluid laden with chemically and/or physically bound carbon dioxide and absorbed into a methanol phase to give a carbon dioxide-laden methanol phase, wherein the carbon dioxide-laden methanol phase is reacted as a carbon dioxide-containing stream with a hydrogen-containing stream in the presence of a catalyst to form the methyl formate, and the methyl formate is reacted with an ammonia-containing stream to form the formamide, or
the scrubbing fluid is an aqueous ammonia solution and carbon dioxide is bound at least partly in the form of carbonates in the scrubbing fluid, wherein the scrubbing fluid laden with chemically and/or physically bound carbon dioxide is reacted as a carbon dioxide-containing stream with a hydrogen-containing stream in the presence of a catalyst to form ammonium formate or to form ammonium formate and formamide, wherein the ammonium formate is converted into the formamide by heat treatment.

22. The process of claim 21 wherein the syngas is a syngas for ammonia synthesis and/or the gas scrubbing is performed on a syngas obtained from steam reforming and/or a subsequent water-gas shift reaction.

23. The process of claim 21 wherein the syngas comprises a gas from a coke oven gas, a blast furnace gas, a converter gas, or an offgas from cement works.

24. The process of claim 21 wherein methanol or an aqueous ammonia solution is used as the scrubbing fluid for the gas scrubbing for removing carbon dioxide.

25. The process of claim 21 wherein either:
the gas scrubbing of the syngas for removing carbon dioxide is performed with the aqueous ammonia solution as the scrubbing fluid at a pressure of 20 to 50 bar and/or at a temperature of below 100° C.; or
the gas scrubbing of the syngas for removing carbon dioxide is performed with methanol as the scrubbing fluid at a pressure of 20 to 50 bar and/or with methanol cooled to a temperature of −20° C. or below.

26. The process of claim 22 wherein the hydrogen-containing stream comprises:
a substream of the syngas after the gas scrubbing;
a substream of the syngas before the gas scrubbing; and
hydrogen obtained from the processing of products of the ammonia synthesis and/or urea synthesis.

27. The process of claim 21 wherein the hydrogen-containing stream comprises hydrogen obtained from processing products of urea synthesis.

28. The process of claim 21 wherein ammonia in the ammonia-containing stream and/or ammonia for the aqueous ammonia solution is obtained from the syngas via ammonia synthesis.

29. The process of claim 21 comprising using a ruthenium-phosphine complex as a catalyst for at least one of:
a reaction of the formamide to form urea or for a reaction of the formamide with ammonia to form urea;
a reaction of the carbon dioxide-laden methanol phase and the hydrogen-containing stream to form the methyl formate; or
a reaction of the aqueous ammonia solution and the hydrogen-containing stream to form the ammonium formate or the ammonium formate and the formamide.

30. The process of claim 29 wherein the ruthenium-phosphine complex comprises at least one monophosphine, one diphosphine, one triphosphine, or one compound having more than three phosphine groups, the phosphine having the formula PR¹R²R³, in which R¹, R², and R³ independently of one another are in each case substituted or unsubstituted alkyl, substituted or unsubstituted aryl, or substituted or unsubstituted heteroaryl.

31. The process of claim 29 wherein the ruthenium-phosphine complex is a ruthenium-triphosphine complex, with the triphosphine having a general formula I: 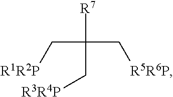
in which R¹ to R⁶ independently of one another are substituted or unsubstituted aryl or substituted or unsubstituted heteroaryl, wherein R⁷ is hydrogen, alkyl, cycloalkyl, or aryl.

32. The process of claim 29 wherein the ruthenium-phosphine complex has a general formula II, (A)Ru(L)₃, in which A is a triphosphine of the general formula I 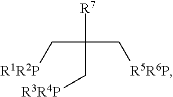
wherein R¹ to R⁶ independently of one another are substituted or unsubstituted aryl or substituted or unsubstituted heteroaryl and R⁷ is hydrogen, alkyl, cycloalkyl, or aryl, wherein L in each case independently of one another are monodentate ligands, wherein two monodentate ligands L are replaceable by one bidentate ligand or wherein three monodentate ligands L are replaceable by one tridentate ligand.

33. The process of claim 21 comprising performing the reaction of the carbon dioxide-laden methanol phase and the hydrogen-containing stream to form the methyl formate and/or the reaction of the carbon dioxide-laden aqueous ammonia solution and the hydrogen-containing stream to form the ammonium formate or the ammonium formate and the formamide using a ruthenium-phosphine complex as the catalyst.

34. The process of claim 21 comprising performing a catalytic reaction of the formamide to form urea or a catalytic reaction of the formamide with ammonia to form urea at a temperature in a range from 50 to 250° C. and/or at a pressure in a range from ambient pressure to 150 bar.

35. The process of claim 34 comprising performing the catalytic reaction of the formamide to form urea or the catalytic reaction of the formamide with ammonia to form urea in a nonpolar or polar aprotic organic solvent or in liquid or supercritical ammonia.

36. The process of claim 21 wherein at least one of:
a catalytic reaction of the carbon dioxide-laden methanol phase and the hydrogen-containing stream to form the methyl formate is performed at a temperature in a range from 20 to 150° C. and/or at a pressure in a range from 40 bar to 220 bar;
a reaction of the methyl formate with ammonia to form the formamide is performed at a temperature in a range from 20° C. to 100° C. and/or at a pressure in a range from atmospheric pressure to 70 bar;
a catalytic reaction of the aqueous ammonia solution laden with chemically bound carbon dioxide and the hydrogen-containing stream to form the ammonium formate or the ammonium formate and the formamide is performed at a temperature in a range from 60 to 180° C. and/or at a pressure in a range from 35 bar to 210 bar; or
a heat treatment of the ammonium formate to form the formamide is performed at a temperature in a range from 100° C. to 185° C.

37. The process of claim 21 comprising reusing methanol formed in a reaction of the methyl formate with the ammonia-containing stream to form the formamide for the scrubbing fluid or a methanol-containing liquid.

38. The process of claim 21 coupled with ammonia synthesis, the ammonia synthesis comprising preparation of the syngas by steam reforming with a water-gas shift reaction, gas scrubbing the syngas with the scrubbing fluid for removing carbon dioxide, methanization of the syngas as scrubbed, and preparation of ammonia with the syngas, wherein the carbon dioxide-laden scrubbing fluid is used as a source of carbon dioxide for the preparation of urea.

39. The process of claim 38 wherein the hydrogen-containing stream comprises a substream of the syngas after the scrubbing, wherein hydrogen is obtained from processing products of the ammonia synthesis and/or urea synthesis, and/or a source of ammonia comprises ammonia formed in the ammonia synthesis.

40. The process of claim 39 wherein the ammonia synthesis comprises a steam reforming in a primary reformer and a downstream secondary reformer and/or a two-stage water-gas shift reaction with a high-temperature shift stage and a low-temperature shift stage.
